(19) United States
(12) Patent Application Publication
(10) Pub. No.: US 2023/0028624 A1
(43) Pub. Date: Jan. 26, 2023

(54) ANTIMICROBIAL COMPOSITIONS

(71) Applicant: OXY SOLUTIONS AS, Oslo (NO)

(72) Inventors: Hege UGLAND, Oslo (NO); Maja KNUTSEN, Oslo (NO); Gary Chinga CARRASCO, Trondheim (NO)

(21) Appl. No.: 17/786,114

(22) PCT Filed: Dec. 16, 2020
(86) PCT No.: PCT/GB2020/053244

§ 371 Date: Jun. 16, 2022

(30) Foreign Application Priority Data
Dec. 16, 2019 (GB) 1918552.9

(51) Int. Cl. A61K 31/717; A61K 47/69; A61K 9/00; A61L 26/00; A61P 17/02; A61P 31/04

(52) U.S. Cl. CPC A61K 31/717; A61K 47/6903; A61K 9/0092; A61L 26/0023; A61P 17/02; A61P 31/04

(57) ABSTRACT

The invention provides antimicrobial compositions comprising charged cellulose nanofibrils dispersed in an aqueous solution having a dissolved oxygen content of at least 20 mg/L, preferably from 20 to 100 mg/L. The cellulose nanofibrils may have an increased surface charge due to their carboxylic acid content which contributes to their antimicrobial properties. In particular, the carboxylic acid content may be at least about 1000 μmol/g cellulose, preferably at least about 1400 μmol/g cellulose. The compositions are suitable for use in the treatment of wounds, in particular chronic wounds.

### TECHNICAL FIELD

The present invention relates to antimicrobial compositions, to methods for their preparation and their medical use.

More specifically, the invention relates to oxygenated nanocellulose compositions which can be used to promote wound healing, and which thus find use in the treatment of wounds. In particular, the compositions may be used in the treatment of biofilm infections present in chronic wounds.

### BACKGROUND OF THE INVENTION

A wound is an injury to the skin accompanied by damage or destruction of the blood supply to skin tissues. This compromises the delivery of oxygen and nutrients required for tissue regeneration. Local wound treatments include wound dressings which may be applied to the wound to provide a barrier to the entry of micro-organisms and protect the wound from the external environment. Some wound dressings also support or promote wound healing mechanisms.

Oxygen, in particular, plays a crucial role in wound healing, including reduction in bacterial infections, increased re-epithelialization, proliferation of fibroblasts, collagen synthesis and angiogenesis. Insufficient oxygenation of wounds due to poor blood circulation impairs proper wound healing and can result in the formation of chronic wounds. Chronic wounds may contain colonies of aerobic and/or anaerobic micro-organisms as part of a biofilm. In 60-100% of chronically open wounds, a biofilm will be present. Bacterial biofilms are common and form when bacteria interact with a body surface to form polymeric films (also known as “exopolysaccharide” or “extracellular polysaccharide” polymers) that coat the body surface and provide a living colony for further bacterial colonisation and proliferation. Bacteria which become lodged in a biofilm are more difficult to remove or kill than those that remain in a plaktonic state (i.e. suspended as single cells) and can be resistant to many antibiotics.

Previous studies have shown that oxygen has an anti-bacterial effect. It has also been suggested that oxygen may play a role in the reduction of biofilm formation. Various oxygen-based therapy approaches to chronic wound treatments are known. These include Hyperbaric Oxygen Therapy (HBOT) and Topical Oxygen Therapy (TOT). HBOT is considered the leading oxygen therapy for chronic wound healing. It involves placing the patient in a pressure chamber and the treatment is based on exposure and breathing pure oxygen gas which is delivered at a pressure greater than ambient pressure. However, this treatment requires specialized equipment and highly skilled personnel which results in a high cost to the healthcare system. TOT is achieved via a sleeve that encases the patient's limb, which is supplied with oxygen gas and pressurized slightly more than atmospheric pressure. However, there is controversy as to the depth of absorption of topical oxygen and therefore its efficacy.

Other approaches to wound treatment include the use of oxygenated dressings. These are inexpensive options for oxygen therapy, however, the number of products currently on the market is limited. Oxygenated dressings either incorporate oxygen predominantly in the form of oxygen gas bubbles or contain components which generate oxygen gas when in use. Examples of such products include the OxyBand™, OxygeneSys™ and Oxyzyme™ dressings.

The OxyBand™ dressing (OxyBand Technologies, Minn., USA) provides for the local delivery of high concentrations of pure oxygen to healing wounds using a directionally permeable, gas-emitting reservoir. The oxygen is stored in a reservoir between an occlusive upper layer and a lower oxygen-permeable film which allows the dressing to supersaturate the wound fluid with oxygen (Lairet et al., J. Burn Care Res. 35(3): 214-8, 2014; Lairet et al., abstract at The Military Health Services Research Symposium, 2012: and Hopf et al., abstract of the Undersea & Hyperbaric Medical Society Annual Scientific Meeting, 2008). The OxygeneSys™ dressing comprises a polyacrylate matrix that forms a closed cell foam structure encapsulating oxygen gas. The walls of the foam cells of the matrix contain dissolved oxygen. When the dressing is moistened with exudate, saline or water the gaseous oxygen within the dressing begins to dissolve into the liquid, but the release rate of oxygen is low and only reaches 15 mg/L (see U.S. Pat. No. 7,160,553). Oxyzyme™ is an enzyme-activated hydrogel dressing system which comprises two polysulphonate sheet hydrogels layered on top of one another. Also contained within the dressing are an oxidase enzyme, glucose and iodide. When removed from its packaging and contacted with a wound, the oxidase enzyme within the top layer is activated upon contact with oxygen in the air and by the contact made between the two layers of the dressing. Reaction of the enzyme with oxygen generates hydrogen peroxide within the dressing which, when it reaches the wound-facing surface, is converted through its interaction with the iodine component of the dressing into dissolved oxygen (Ivins et al., Wounds UK, Vol. 3 No. 1, 2007; and Lafferty et al., Wounds UK, Vol. 7 No. 1, 2011).

Oxygenated dressings represent an improvement in the delivery of topical oxygen to the wound environment over the hyperbaric chamber and have shown encouraging results in case studies (see, for example, Lairet et al., 2014; Lairet et al., 2012; Hopf et al., 2008; Ivins et al., 2007; and Lafferty et al., 2011 (all as above); Roe et al., Journal of Surgical Research 159: e29-e36, 2010; Zellner et al., Journal of International Medical Research Vol. 43(1), 93-103, 2014; and Kellar et al., Journal of Cosmetic Dermatology 12: 86-95, 2013). However_{;} documentation of the oxygen concentration/availability and oxygen stability of these products is limited and these are not in widespread use.

Recent studies have shown that dissolved oxygen diffuses and penetrates tissue more efficiently compared to directly exposing the tissue to oxygen gas (see e.g. Roe et al., 2010 (as above), Stüker, J. Physiol. 538(3): 985-994, 2002; Atrux-Tallau et al., Skin Pharmacol. Physiol. 22: 210-217, 2009; Reading et al., Int. J. Cosmetic Sci. 35:600-603, 2013; and Charton et al., Drug Design; Devel. and Ther. 8:1161-1167, 2014), None of the existing therapies enables the direct delivery of high levels of dissolved oxygen to wound tissues. For the most part, these deliver oxygen in the form of a gas which must dissolve (e.g. in wound exudate or other cellular fluid) before it can be effective. This limits the efficacy of the treatment. Although the OxygeneSys™ dressing contains some dissolved oxygen in the moisture which coats the walls of the foam matrix, the release rate of oxygen only reaches a maximum of 15 mg/L. Other treatments which are able to provide a high level of oxygen directly to the tissues in dissolved form would thus be beneficial for use in the treatment of wounds.

Nano-structured cellulose (“nanocellulose”) is a well-known material which can be produced from various cellulose sources such as wood pulp. Cellulose nanofibrils (“CNF”) are one type of nanocellulose. These comprise nanoscale cellulose fibrils having a high aspect ratio, with widths (i.e. diameters) on the nanometer scale and lengths on the micrometer scale. The fibrils can be isolated from cellulose-containing materials, such as wood-based fibres, by various mechanical methods such as high velocity impact homogenization, grinding or microfluidization.

CNF materials have been suggested for various uses in the biomedical field. This includes use as scaffolds for tissue regeneration, as wound dressings, as carriers for antimicrobial components and as bio-inks for 3D printing. In the production of such materials, chemical pre-treatment methods such as 2,2,6,6-tetramethyl piperidinyl-1-oxyl (TEMPO)-mediated oxidation have been proposed to adjust their properties. TEMPO-CNF has negatively charged carboxyl groups at physiological pH values. A minor fraction of aldehydes is also produced during TEMPO-mediated oxidation. TEMPO-CNF forms a gel with high viscosity when provided at low concentrations in water. Such gels comprise nanofibrils arranged in a hydrogel network which has good water holding capacity and mechanical properties that resemble the texture of soft tissue. This, together with their antimicrobial activity and ability to form translucent structures, has led to their proposed use in the development of wound dressing materials (see Powell et al., Carbohydrate Polymers 137(10); 191-197, 2016; and Jack et al., Carbohydrate Polymers 157: 1955-1962, 2017).

Previously, it has been demonstrated that TEMPO-CNF in gel form inhibits growth of the wound pathogen Pseudomonas aeruginosa (Powell et al., 2016; and Jack et al., 2017—both as above). Antimicrobial inhibition of the TEMPO-CNF gel was found to be concentration dependent, i.e. the higher the concentration, the higher the inhibition of growth of P. aeruginosa. This was partly attributed to a limitation in mobility of the bacteria (Jack et al., 2017—as above). This has been confirmed in a more recent study where TEMPO-CNF from the same pulp fibre inhibited bacterial swimming potential of the food pathogens B. cereus, verotoxigenic E. coil, L. monocytogenes and S. Typhimurium (Silva et al,, J. Mater. Sci. 54(18), 12159-12170, 2019). However, to date there has been no recognition that the antimicrobial activity of CNF materials may be influenced by their surface properties.

A need still exists for alternative materials which can be used to treat wounds, especially chronic wounds associated with biofilm infections. In particular, there is a need for such materials which provide a cost-effective treatment, which are easy to use, and which can be used to effectively treat wounds with minimal inconvenience to the subject being treated (e.g. a patient).

### SUMMARY OF THE INVENTION

The inventors have now found that the antimicrobial activity of CNF materials is dependent on their surface properties and may be enhanced by increasing their surface charge. When provided as a low concentration dispersion in an aqueous solution, they have also found that such materials can be effectively oxygenated to further potentiate their antimicrobial activity. The inventors therefore propose oxygenated nanocellulose-based compositions containing cellulose nanofibrils which have high surface charge and the use of such compositions in the treatment of wounds, in particular chronic wounds.

The compositions herein disclosed contain cellulose nanofibrils having a high surface charge and are oxygenated such that they have high levels of dissolved oxygen. They can be provided in “ready-to-use” form, or they can be prepared at the point of use. For example, the compositions may be provided as an oxygenated “liquid” (which includes thickened or ‘viscous’ liquids), or they may be provided in the form of an oxygenated gel (i.e. a “hydrogel”) which contains the charged cellulose nanofibrils. Such compositions can be used directly at a wound site, or they may be incorporated into a suitable wound covering, such as a bandage, gauze, patch or absorptive pad, etc. The oxygenated gels can also be 3D printed for use as a wound dressing, or such gels may be prepared at the point of use from a nanofibrillated cellulose aerogel.

Due to their antimicrobial activity, the compositions are particularly suitable for use in the treatment of infected wounds and can readily be delivered to a wound site, either by direct application to the affected tissues or by incorporation into a suitable wound covering which is intended to be applied to the desired target site. For example, the compositions may be provided in, or as a component of, a wound covering such as a bandage, gauze, patch or absorptive pad for application to the target site.

In one aspect the invention provides an antimicrobial composition comprising charged cellulose nanofibrils dispersed in an aqueous solution, wherein said solution has a dissolved oxygen content of at least 20 mg/l.

In another aspect the invention provides a composition as herein described for use as an antimicrobial agent, for example for use in inhibiting the growth of at least one wound pathogen.

In another aspect the invention provides a method for the preparation of a composition as herein described, said method comprising the following steps: (i) providing a dispersion of charged cellulose nanofibrils in an aqueous solution; and (ii) oxygenating said dispersion.

In another aspect the invention provides a method for treating a wound, said method comprising the step of applying an effective amount of an antimicrobial composition as herein described to said wound. Optionally, said method may further comprise the step of applying a wound covering (herein referred to as a “secondary dressing”) following application of said antimicrobial composition.

In another aspect the invention provides the use of an antimicrobial composition as herein described in the manufacture of a medicament for use in a method for treating a wound.

In another aspect the invention provides a kit for use in treating a wound, the kit comprising: (a) a sterilised, sealed container or package containing an antimicrobial composition as herein described; and (b) a wound covering, e.g. a wound dressing, bandage, gauze, patch or absorptive pad. The kit may additionally comprise printed instructions for use of the components of the kit in the treatment of a wound.

In another aspect the invention provides a kit for use in treating a wound, the kit comprising: (a) a sterilised, sealed container or package containing an aerogel comprising charged cellulose nanofibrils; and (b) an oxygenated aqueous liquid (e.g. oxygenated water or oxygenated saline) having a dissolved oxygen content of at least 20 mg/l. The kit may additionally comprise printed instructions for mixing of the components whereby to form an oxygenated hydrogel and its use in the treatment of a wound.

In another aspect, the invention provides a wound covering, e.g. a bandage, gauze, patch or absorptive pad, having incorporated therein an antimicrobial composition as herein described.

In another aspect, the invention provides a wound dressing in the form of a hydrogel comprising charged cellulose nanofibrils, wherein said hydrogel has a dissolved oxygen content of at least 20 mg/l. The wound dressing may be a 3D printed hydrogel.

### DETAILED DESCRIPTION OF THE INVENTION

### Definitions

The terms “nanofibrillar cellulose” and “cellulose nanofibrils” are used interchangeably herein and refer to isolated cellulose fibrils or fibril bundles derived from cellulose material. The cellulose fibrils are characterised by a high aspect ratio (i.e. length:diameter). Their length may exceed 1 μm, but their diameter is in the submicron range, i.e. less than 1 μm. Typically, their diameter will be on the nanometer scale. When dispersed in an aqueous solvent (e.g. water), the cellulose fibrils or fibril bundles have the ability to form a viscoelastic gel (i.e. a hydrogel) at low concentrations. As will be understood, the actual concentration for gel formation will be dependent on other factors, such as the precise nature of the nanofibrillar cellulose, for example its degree of fibrillation.

The terms “oxidised cellulose nanofibrils” and “oxidised CNFs” are used interchangeably herein and refer to surface-oxidised cellulose nanofibrils in which at least a proportion of the primary hydroxyl groups present in the native cellulose material have been oxidised to aldehyde and/or carboxyl groups. “Oxidised cellulose nanofibrils” include, but are not limited to, TEMPO-mediated oxidised cellulose nanofibrils (also referred to herein as “TEMPO-CNFs”).

As used herein, the term “gel” refers to a form of matter that is intermediate between a solid and a liquid. It is self-holding yet deformable. A gel is generally resistant to flow at ambient temperature, i.e. at a temperature below about 25° C., preferably below about 20° C. In rheological terms, a “gel” may be defined according to its storage modulus (or “elastic modulus”), G′, which represents the elastic nature (energy storage) of a material, and its loss modulus (or “viscous modulus”), G″, which represents the viscous nature (energy loss) of a material. Their ratio, tan δ (equal to G″/G′), also referred to as the “loss tangent”, provides a measure of how much the stress and strain are out of phase with one another. A “gel” has a loss modulus (G″) which is less than its storage modulus (G′) and a loss tangent (tan δ) which is less than 1.

The term “viscoelastic” when used in relation to a gel means that the gel is characterised by rheological properties which resemble, in part, the rheological behaviour of a viscous fluid and, also in part, that of an elastic solid.

The term “hydrogel” when used in relation to a gel means that the gel is hydrophilic and contains water.

As used herein, the term “aerogel” refers to a porous material derived from a gel in which the liquid component of the gel is replaced with a gas (typically air), An “aerogel” is a solid having an extremely low density,

Unless otherwise defined, the term “liquid” as used herein refers to a substance which flows freely and which maintains a constant volume. It includes thickened liquids and viscous liquids which flow. A “liquid” will typically have a loss modulus (G″) which is greater than its storage modulus (G′) and a loss tangent (tan δ) which is greater than 1.

The term “viscosity” when used in relation to a substance is the extent to which the substance is resistant to flow when subjected to stress. Viscosity may refer to Brookfield viscosity which is measured using a Brookfield viscometer. For example, viscosity may be measured using a Brookfield DV2TRV viscometer operated under the following parameters: assessed volume of substance: 200 ml; temperature: 23° C.±1° C.; spindles: V-71; speed (shear rate): 10 RPM.

The term “wound covering” as used herein means any material intended to be applied to a body tissue or body surface and which is intended to remain in place to aid in wound healing. It encompasses materials such as wound dressings, bandages, gauzes, patches, plasters, absorptive pads, etc. In some embodiments the invention relates to such a wound covering which incorporates an oxygenated nanocellulose composition as herein described (e.g. in liquid, thickened liquid, or gel form). In other embodiments, such a wound covering may be applied to a wound site following application of an antimicrobial composition as herein described.

The term “wound” includes any defect or disruption in the skin which may result from physical, chemical or thermal damage, or as a result of an underlying medical or physiological condition. A wound may be initiated in a variety of ways, for example it may be induced by trauma, cuts, ulcers, burns, surgical incisions, etc. A wound may be classified as acute or chronic.

The term “bacterial biofilm” means a community of bacteria which are contained within an extracellular polymeric substance (EPS) matrix produced by the bacteria and attached to a body surface.

The term “antimicrobial” when used in relation to a substance means that the substance can kill, inhibit or control the growth of at least one micro-organism, for example a bacterial organism such as, but not limited to, any of the following: Pseudomonas aeruginosa, Staphylococcus aureus, Streptococcus epidermis and Escherichia coli.

In one aspect the invention provides antimicrobial compositions comprising charged cellulose nanofibrils dispersed in an aqueous solution, wherein said solution has a dissolved oxygen content of at least 20 mg/l.

Depending on the concentration of cellulose nanofibrils and their degree of fibrillation, such compositions may be provided in the form of liquids (e.g. viscous liquids), or they may be provided as hydrogels. As hydrogels these contain water which is trapped or immobilised within the three-dimensional network provided by the fibrils of cellulose. In the compositions herein disclosed the water acts as a carrier for the oxygen.

The compositions disclosed herein are antimicrobial and, when applied to a wound, can aid in healing, regeneration or restoration of a normal metabolic state. They are convenient to apply to the target tissue irrespective of its size and location and are capable of the release of dissolved oxygen directly at the point of contact with the body tissues. The compositions may be used as such and so applied directly to the body tissues, or these may be used in conjunction with other wound coverings. For example, they may be incorporated into or form part of a suitable “wound covering” which is intended to be applied to the wound. In some cases, the antimicrobial compositions may be provided in, or as a component of, a wound dressing, bandage, gauze, patch, plaster, absorptive pad, or any other wound covering which is suitable for application to the target site.

The antimicrobial compositions are conveniently applied to the desired target site either alone or in conjunction with other wound coverings. They are able to make intimate contact with the target tissues and can deliver active oxygen in a controlled manner to effectively kill, inhibit or control the growth of micro-organisms. Their antimicrobial activity is further potentiated by the charged cellulose nanofibrils which, in the case of a gel, form the three-dimensional network of the hydrogel structure. Specifically, the inventors have found that antimicrobial activity is enhanced where the cellulose nanofibrils have a high surface content of carboxylic acid and/or aldehyde groups, for example a surface carboxylic acid group content of at least about 1000 μmol per g of cellulose, preferably at least about 1400 μmol per g of cellulose, and/or a surface aldehyde group content of at least about 100 μmol per g of cellulose, preferably at least about 200 μmol per g of cellulose. Due to their water content, the compositions of the invention also usefully serve to moisturise the target tissues.

The compositions herein described comprise the cellulose nanofibrils dispersed in an aqueous solution which contains high levels of dissolved oxygen. As will be understood, the aqueous solution will be physiologically tolerable. The aqueous solution contains water, but it need not be pure water and may contain other physiologically tolerable components. For example, the aqueous solution may be saline such as phosphate buffered saline (PBS).

The cellulose nanofibrils which are present in the compositions of the invention are surface-charged. They may carry positive or negative surface charge, but preferably they carry negative charge, i.e. they are anionic. In one embodiment, the cellulose nanofibrils are “oxidised”, i.e. these have been chemically modified by oxidation of at least a proportion of the primary hydroxyl groups present in the native cellulose material to carboxyl groups and/or aldehyde groups.

Chemical modification will typically be carried out in respect of the fibrous cellulose raw material prior to its disintegration into nanofibrils, i.e. prior to “fibrillation”. For example, it may be carried out in respect of the fibrous cellulose raw material when provided as a dispersion in water, i.e. when it is provided as a “pulp”. The oxidized cellulose pulp may then be subjected to fibrillation as herein described.

Chemical modification involves modifying the chemical structure of the cellulose by a chemical reaction or reactions. The cellulose material for use in the invention may be oxidised to modify the functional groups of the cellulose molecule. Specifically, oxidation is effective to convert a proportion of the primary hydroxyl groups of the cellulose to aldehydes and/or carboxyl groups. Oxidation also includes carboxymethylation in which a proportion of the hydroxyl groups are converted to carboxymethyl groups, and phosphorylation in which some or all of the hydroxyl groups are phosphorylated.

The extent of chemical modification will be dependent on the choice of chemical for pre-treatment, its concentration and the reaction conditions. The extent of chemical modification may be varied as required. As described herein, a higher level of oxidation may be beneficial to enhance antimicrobial activity.

The hydroxyl groups of the cellulose may be oxidised catalytically, for example using a heterocyclic nitroxyl compound. Any heterocyclic nitroxyl compound capable of catalysing the selective oxidation of the hydroxyl groups of the C6 carbon in cellulose may be used. In one embodiment, the heterocyclic nitroxyl compound may be 2,2,6,6-tetramethylpiperidinyl-1-oxy free radical (generally known as “TEMPO”), or any derivative thereof (see lsogai et al., Nanoscale 3:71, 2011). In one embodiment, the cellulose for use in the invention is “TEMPO-oxidised cellulose”.

Suitable oxidizing agents include, but are not limited to, hypohalites (e.g. sodium hypochlorite), sodium chlorite and periodate. Combinations of such agents may also be used. Hypohalites, such as sodium hypochlorite, are suitable for use in the production of oxidised cellulose materials having a proportion of both carboxyl groups and aldehydes. Sodium chlorite may be used in cases where the conversion of substantially all hydroxyl groups to carboxyl groups is desired. For example, it may be used after TEMPO-mediated oxidation to convert the remaining aldehyde groups to carboxyl groups. Periodate oxidation provides modified cellulose materials having a proportion of 2,3-dialdehyde units along the polymer chain by selective cleavage between the C2 and C3 (see Liimatainen et al., Biomacromolecules 5(5): 1983-1989, 2004). To provide the desired increase in charge, periodate may be used in combination with other oxidizing agents such as sodium chlorite, or in combination with carboxymethylation or TEMPO-mediated oxidation to introduce carboxyl groups in the C6 position (see Chinga-Carrasco et al., Journal of Biomaterials Applications 29(3): 423-432, 2014). The use of hypohalites in TEMPO-mediated oxidation is generally preferred for use in preparing the nanocellulose materials for use in the invention. Methods for carboxymethylation and phosphorylation are well known in the art and described, for example, in Wågberg et al., Langmuir 24: 784-795, 2008, Chinga-Carrasco et al., Journal of Biomaterials Applications 29(3): 423-432, 2014, and Ghanadpour et al., Biomacromolecules 16: 3399-3410, 2015, the contents of which are incorporated herein by reference.

As a result of oxidation, the primary hydroxyl groups (i.e. the C6 hydroxyl groups) of the cellulosic β-D-glucopyranose units are selectively oxidised to carboxylic acid groups. Some of the primary hydroxyl groups may be only partially oxidised to aldehyde groups. The content of carboxylic acid groups in the cellulose material may be determined by methods known in the art, for example using conductometric titration as described by Saito et al. in Biomacromolecules 5(5): 1983-1989, 2004. The content of aldehyde groups may similarly be determined using methods well known in the art, for example by spectrophotometric methods such as described by Jausovec et al. in Carbohydrate Polymer 116:74-85, 2015. Carboxylic acid and aldehyde levels in the cellulose may be defined in terms of μmol per g of cellulose material.

Different degrees of oxidation of the cellulose material can be achieved, for example using different chemical pre-treatment agents and/or by varying the concentration of such agents. As evidenced herein, the inventors have surprisingly found that an increase in charge in the nanocellulose material (i.e. an increase in the degree of oxidation) can impact its antimicrobial properties.

In some embodiments, the carboxylic acid content of the oxidized cellulose may range from 400 to 1750 μmol/g cellulose, preferably from 700 to 1700 μmol/g cellulose, e.g. from 800 to 1600, from 900 to 1600, or from 1000 to 1600 μmol/g cellulose. In certain embodiments, the carboxylic acid content may be at least about 1000 μmol/g cellulose, preferably at least about 1400 μmol/g cellulose, for example it may range from 1400 to 1700 μmol/g cellulose, e.g. from 1500 to 1600 μmol/g cellulose. In certain embodiments, the carboxylic acid content of the oxidized cellulose material may be greater than 900 μmol/g, preferably greater than 1000 μμmol/g, e.g. greater than 1400 μmol/g cellulose.

In some embodiments, the aldehyde content of the oxidized cellulose may range from 10 to 1700 μmol/g cellulose, preferably from 100 to 400 μmol/g cellulose, e.g. from 200 to 400 μmol/g cellulose. In certain embodiments, the aldehyde content may be less than 300 μmol/g cellulose, for example less than 250 μmol/g cellulose. In other embodiments, the aldehyde content may be at least 300 μmol/g cellulose.

In certain embodiments, the oxidized cellulose may have a carboxylic acid content of at least about 1400 μmol/g cellulose, e.g. from 1400 to 1700 μmol/g cellulose or from 1500 to 1600 μmol/g cellulose, and an aldehyde content of less than 300 μmol/g cellulose, e.g. less than 250 μmol/g cellulose.

The presence of carboxylic acid groups in the cellulose molecules after chemical modification (and thus anionic charge at physiological pH) may also be beneficial since it decreases the extent of hydrogen bonding between the cellulose fibres and so aids in the disintegration process (i.e. fibrillation) to produce nanofibrillar cellulose. It also provides a nanofibrillar cellulose material with high viscosity even at low concentrations.

In one embodiment, the raw cellulose material may be subjected to a pre-treatment prior to oxidation. For example, it may be autoclaved in the presence of an alkali material such as sodium hydroxide. Such treatment serves to remove endotoxins (i.e. lipopolysaccharides, LPS) and may be carried out as described by Nordli et al. in Carbohydrate Polymers 150: 65-73, 2016, the entire content of which is incorporated herein by reference. The content of LPS will typically be less than about 100 endotoxin units per g of cellulose to be considered ultrapure for wound dressing applications. Alkali treatment also serves to reduce the lignin content of the cellulose. This will generally be less than 1 wt. % of the cellulose material.

The nanofibrillar cellulose may be prepared from raw cellulose material of any origin, though typically it will be prepared from cellulose material of plant origin. It may be derived from any plant material that contains cellulose, for example from wood or a plant. Other cellulose raw materials include those derived from bacterial fermentation processes. Cellulose may also be obtained from algae or tunicates.

In one embodiment, the cellulose material of plant origin is wood. Wood may be obtained from any softwood or hardwood tree. Softwood trees which are suitable include spruce, pine, fir, larch and hemlock. Hardwood trees which are suitable include birch, aspen, poplar, alder, oak, beech, acacia, and eucalyptus. Mixtures of wood from soft and hardwood trees may also be used.

In one embodiment, the cellulose-containing material is obtained from wood-derived fibrous material. Typically, it will be derived from wood pulp, i.e. from a combination of the wood-derived fibrous material in water. Wood pulp is formed by the chemical or mechanical separation cellulose fibres from wood. The cellulose-containing material may be obtained from softwood pulp, for example from pulp derived from pine. In one embodiment, the softwood may be Pinus radiata, also known as Monterey pine or radiata pine, which is a fast growing medium density softwood. In another embodiment, it may be Pinus Sylvestris. In another embodiment the softwood may be a spruce, for example a Picea species. In another embodiment the cellulose material may be obtained from hardwood pulp,

Raw cellulose materials are composed mainly of cellulose, hemicelluloses and a smaller amount of lignin. The cellulose materials may be obtained through kraft and/or sulphite processes. In some embodiments, the natural cellulose material may be pre-treated in order to remove (either completely or partially) matrix materials such as lignin to provide a purified cellulose material. Bleached wood pulp is an example of such a purified material. Bleaching may be carried out using conventional bleaching methods, such as an Elemental Chlorine Free (ECF) process or totally Chlorine Free (TCF) bleaching process.

Fibrillation of cellulose to produce cellulose nanofibrils may be carried out using known methods such as homogenization of aqueous dispersions of the chemically modified cellulose fibres (e.g. pulp fibres) as herein described. Even at very low concentrations, the resulting dispersion of cellulose nanofibrils is a dilute viscoelastic hydrogel.

In the preparation of nanofibrillar cellulose, cellulose fibres are disintegrated to produce fibrils having a sub-micron diameter. For example, these may have a diameter which is in the nanometer range.

Disintegration methods include mechanical disintegration of the cellulose material in the presence of water. Mechanical disintegration may involve grinding, crushing, or shearing of the fibrous cellulose material or any combination of these. It may be carried out using known equipment such as a homogenizer, fluidizer (e.g. a microfluidizer), grinder, etc. In one embodiment, disintegration may be carried out using a homogenizer in which the fibrous material is subjected to homogenization under pressure. Forcing the fibrous material through a narrow opening under pressure gives rise to an increase in velocity and thus shearing forces which result in separation of the individual fibrils or fibril bundles from the cellulose material. Where appropriate, several stages of mechanical disintegration may be carried out in order to achieve the desired degree of fibrillation. For example, when using a homogenizer, several passes through the homogenizer may be required. An example of a homogenizer which may be used to effect fibrillation is the Rannie 15 type 12.56X homogenizer.

Following fibrillation, the resulting cellulose nanofibrils or nanofibril bundles are characterised by a high aspect ratio (i.e. length : diameter). Their length may exceed 1 μm, but their diameter is in the submicron range, i.e. less than 1 μm. Precise dimensions and size distribution of the nanofibrils or nanofibril bundles will depend on the nature of the raw cellulose material and the disintegration (i.e. fibrillation) method and may vary to some extent. Chemical modification of the cellulose may also affect the fibril size and fibril size distribution. For example, TEMPO-mediated oxidation may produce fibrils or fibril bundles having a reduced length and/or a reduced diameter. The precise dimensions are not considered critical to the invention.

Typically, the diameter of the nanofibrils or nanofibril bundles will be on the nanometer scale, for example less than 20 nm. For example, their average diameter may range from 3 to 20 nm, preferably from 5 to 20 nm, e.g. from 5 to 10 nm. TEMPO-CNFs may have reduced diameters, for example these may have an average diameter in the range from 1 to 10 nm.

Typically, the average length of the nanofibrils or nanofibril bundles will be in the range from 5 to 10 μm. For example, it may be in the range from 1 to 5 μm, e.g. 0.5 to 1 μm, or 0.2 to 0.5 μm.

Size and size distribution of the fibrils may be determined using known techniques, for example by microscopy. Length and diameter may be determined by analysis of images from a scanning electron microscope (SEM), transmission electron microscope (TEM), or an atomic force microscope (AFM). Atomic force microscopy is particularly suitable for measuring the diameter of the fibrils and may, for example, be performed using a Veeco multimode V operated at ambient temperature with AFM tips having a spring constant of about 0.4 Nm⁻¹. TEM may be used for measuring the length.

The nanofibrillar cellulose material may be characterised in terms of the viscosity of an aqueous solution in which it is dispersed. Viscosity may be measured using conventional methods and apparatus. Viscosity may refer to Brookfield viscosity which is measured using a Brookfield viscometer. A number of Brookfield viscometers are commercially available and may be used to measure viscosity. For example, a Brookfield viscometer DV2TRV may be used. When using this apparatus, the following parameters may be used: assessed volume of substance: 200 ml; temperature: 23° C.±1° C.; vane spindle: V-71; speed (shear rate): 10 RPM.

The viscosity of the compositions herein described may be suitable adjusted, for example by varying the concentration of the nanofibrillar cellulose material, its degree of fibrillation, etc. In one embodiment, the viscosity of the compositions may be determined as the Brookfield viscosity. Generally, the Brookfield viscosity of the compositions may range from 20 to 20,000 mPa·s (when measured at 10 RPM, and at a temperature of 23° C.).

In one embodiment, a 0.2 wt. % dispersion of the cellulose nanofibrils in an aqueous solution may provide a composition having a Brookfield viscosity in the range from 20 to 600 mPa·s, preferably 100 to 200 mPa·s, e.g. 200 to 400 mPa·s, or 400 to 600 mPa·s (when measured at 10 RPM, 23° C.). When provided as a 0.4 wt. %

dispersion in an aqueous solution, the cellulose nanofibrils may provide a composition having a Brookfield viscosity in the range from 1500 to 9000 mPa·s, preferably from 1500 to 6000, e.g. 3000 to 6000 mPa·s (when measured at 10 RPM, 23° C.). At a concentration of about 0.5 wt. %, a dispersion of the cellulose nanofibrils in an aqueous composition may provide a Brookfield viscosity in the range from 10,000 to 20,000 mPa·s, preferably 10,000 to 15,000 or 15,000 to 20,000 mPa·s (when measured at 10 RPM, 23° C.).

As a result of the process used to produce the cellulose nanofibrils, the resulting cellulose material may also comprise a proportion of non-nanofibrillar pulp, i.e.

residual cellulose fibres. However, if present, it will typically be present as a minor fraction. The amount of non-nanofibrillar pulp which may be present in the compositions herein described may range from 1 to 20 wt. %, e.g. from 1 to 5 wt. % (based on the total dry weight of cellulose). Total cellulose as referred to herein refers to the dry weight of the total cellulose in the material. In one embodiment, the material will be substantially free from non-nanofibrillar pulp. For example, the amount of non-nanofibrillar pulp may be 0 wt. %.

The content of cellulose nanofibrils in the compositions herein described may range from 0.1 to 1.0 wt. %, preferably from 0.2 to 0.8 wt. %, e.g. from 0.3 to 0.5 wt. %

based on the total weight of the composition. In some embodiments, it may range from 0.5 to 1.0 wt. %.

The materials according to the invention comprise chemically modified nanofibrillar cellulose as described herein. However, they may also contain a proportion of non-modified nanofibrillar cellulose.

As will be understood, depending on the cellulose raw material used to produce the nanocellulose fibrils, the materials herein described may also contain other non-cellulose components. For example these may contain other wood components such as lignin or hemi-cellulose. The nature and amount of such components will be dependent on the cellulose source and method used to prepare the nanocellulose fibrils. When present, these will be present in relatively low amounts, for example less than about 1 wt. % lignin and less than about 20 wt. % hemicellulose, based on the total weight of the composition.

The compositions herein disclosed contain dissolved, molecular oxygen and are capable of releasing this to the target tissues following application to the wound. Since this is intended to function as an active and to deliver a certain level of oxygen to the tissues, its concentration should be chosen accordingly. The precise oxygen level will depend on various factors, including the precise nature of the composition (e.g. any other components which may be present and their stability in the presence of oxygen), the intended use and duration of any treatment, the patient to whom the composition is to be administered, etc. Suitable levels may readily be determined by those skilled in the art according to need.

The compositions herein described contain at least about 20 mg/l dissolved oxygen. In some embodiments they may contain from 20 to 100 mg/L oxygen, from 20 to 70 mg/L, from 20 to 60 mg/L, from 25 to 50 mg/L, or from 30 to 40 mg/L. Compositions comprising elevated levels of oxygen, for example at least 25 mg/L or at least 30 mg/L, are particularly preferred. In one set of embodiments, dissolved oxygen levels may range from 20 to 55 mg/L, e.g. from 25 to 50 mg/L, from 25 to 40 mg/L, or from 30 to 35 mg/L. Oxygen content may be determined using an Orion RDO Oxygen meter (Orion A323, Thermo Scientific, Mass., USA). Unless otherwise specified, all oxygen contents referred to herein are measured at ambient temperature, e,g, in the range 18 to 23° C. It will be understood that all oxygen contents referred to herein are measured at atmospheric pressure.

The wound healing process involves various overlapping stages in which a variety of cellular and matrix components act together to re-establish integrity of damaged tissue and replacement of lost tissue. These are generally considered to involve: haemostasis, inflammation, migration, proliferation and maturation phases. Acute hypoxia stimulates angiogenesis, whereas raised tissue oxygen levels stimulate epithelialisation and fibroblasts. Different concentrations of oxygen may be employed during the different stages of wound healing.

The oxygen present n the compositions according to the invention is dissolved in an aqueous medium which is physiologically tolerable, for example a physiological salt solution (e.g. saline) or water. Typically this will be water.

A number of different methods may be used to prepare the antimicrobial compositions according to the invention. The precise method of preparation may be varied taking into account factors such as the nature of the components and the form of the final product, for example whether this is a liquid or a gel. The step of oxygenation may be carried out in respect of one or more liquid components of the compositions prior to preparation of the final cellulose-containing composition, or it may be carried out in respect of the final composition. As will be described, it is possible to oxygenate thickened liquids or gels (where these are flowable) using known oxygenation methodology. Any of the methods herein described for the preparation of the antimicrobial compositions form further aspects of the invention.

In certain embodiments, the antimicrobial compositions may be prepared by combining an aqueous solution containing dissolved oxygen with a preparation which contains the charged cellulose nanofibrils. For example, a highly oxygenated solution (e.g. water or saline) may be combined with an aqueous dispersion containing the cellulose nanofibrils (e.g. a hydrogel containing the nanofibrilla ed material). Alternatively, an oxygenated solution may be contacted with an aerogel containing the charged cellulose nanofibrils whereby to re-hydrate the aerogel and form a hydrogel.

In a further aspect, the invention thus provides a method for the preparation of an antimicrobial composition as herein described, said method comprising the step of combining an aqueous solution having a dissolved oxygen content of at least 20 mg/l with a preparation which contains charged cellulose nanofibrils.

In other embodiments, the antimicrobial compositions according to the invention may be prepared by oxygenating an aqueous solution in which the charged cellulose nanofibrils are dispersed. In this case, the aqueous solution for oxygenation containing the cellulose material may be provided in the form of a liquid or a flowable gel.

Aqueous solutions containing high levels of dissolved oxygen and methods for heir preparation are generally known in the art. Examples of such solutions and methods for their preparation are described in WO 02/26367, WO 2010/077962 and WO 2016/071691, the entire contents of which are incorporated herein by reference. These solutions may be employed in preparing the antimicrobial compositions herein described. The OXY BIO System (Oxy Solutions, Oslo, Norway) may be used to produce any of the oxygenated solutions herein described.

In one embodiment, aqueous solutions containing high levels of dissolved oxygen and which may be used in preparing the compositions of the invention can be produced by a method which comprises the following steps:
introducing a pressurized liquid (e.g. water) into a piping network to form a flow stream;
injecting gaseous oxygen into the flow stream to produce a mixture of liquid and oxygen bubbles,
providing a linear flow accelerator including a venturi; and
passing the flowing mixture of liquid and gaseous oxygen bubbles through the linear flow accelerator to accelerate the flowing mixture and to subsequently decelerate the flowing mixture to subsonic speed to break up the gaseous oxygen bubbles.

Oxygenation using the above method makes it possible to produce an oxygenated liquid (e.g. water) having a high and stable dissolved oxygen content. When the liquid is water, the solubility of oxygen is increased from about 7 mg/l to 20, 30 50, 60, 70 mg/l or more, and the oxygen content is substantially stable in a cooled environment for months.

This method may further comprise the step of introducing the liquid into a holding volume (e.g. a holding tank) as described in WO 2016/071691. The liquid may be introduced into the holding volume prior to the formation of the liquid and oxygen mixture, or it may be introduced into the holding volume downstream of the venturi. The holding volume may be pressurised, but it need not be. The liquid in the holding tank may, if required, be agitated to maintain the homogeneity of the liquid. In a preferred embodiment, the holding volume is in fluid communication with and downstream of the outlet, and preferably also in fluid communication with and upstream of the liquid inlet of the apparatus, e.g. via appropriate conduits.

In some embodiments, the liquid for oxygenation may further contain one or more foam reducing agents (e.g. simethicone), or the method may comprise an additional foam reducing step. The foam reducing step may comprise any suitable and desired method and it may be provided at any suitable point in the oxygenation method. In one embodiment, the foam reducing step may comprise introduction of the liquid into a holding volume (e.g. a holding tank) as herein described.

Apparatus suitable for carrying out such oxygenation methods may comprise:
a liquid inlet for supplying a liquid (e.g. water) into the apparatus;
an oxygen inlet for supplying oxygen into the liquid within the apparatus to create a liquid and oxygen mixture, the oxygen inlet being in fluid communication with, and downstream of, the liquid inlet;
a venturi in fluid communication with, and downstream of, the liquid inlet and the oxygen inlet, wherein the venturi is arranged to dissolve the oxygen into the liquid passing through the venturi; and
an outlet for the oxygenated liquid in fluid communication with, and downstream of, the venturi.

This apparatus comprises liquid and oxygen inlets and an outlet, with a venturi therebetween. Liquid and oxygen are supplied into the apparatus via the respective inlets, the oxygen inlet being positioned downstream of the liquid inlet such that the oxygen is injected into the liquid stream. This liquid and oxygen mixture is then passed to a venturi, e.g. via a conduit in fluid communication with, and downstream of, the liquid inlet and the oxygen inlet, the conduit being arranged to supply the liquid and the oxygen to the venturi. Owing to the restriction the venturi creates in the flow path, this causes the liquid and oxygen mixture to accelerate through the venturi and then decelerate at the other side, generating a shockwave in the mixture which forces the oxygen to dissolve in the liquid, thus oxygenating the liquid.

In one embodiment the apparatus comprises a diffusion chamber in fluid communication with, and downstream of, the oxygen inlet (and also the liquid inlet), the diffusion chamber and the oxygen inlet being arranged such that the oxygen is supplied through the oxygen inlet into the diffusion chamber. The diffusion chamber provides a volume through which the liquid flows and into which the oxygen is injected, with the diffusion chamber being arranged to promote the break-up of bubbles of oxygen into smaller bubbles, e.g. by encouraging turbulent flow of the liquid and the oxygen in the diffusion chamber. Preferably a grid or mesh, e.g. made from glass, metal or plastic, is arranged in the diffusion chamber, e.g. through which the oxygen and liquid must pass into the diffusion chamber. This helps to break-up the oxygen into small bubbles within the liquid so that they are more easily dissolved into the liquid in the diffusion chamber and downstream in the apparatus, e.g. in the venturi.

The apparatus may comprise a mixing chamber in fluid communication with, and downstream of, the oxygen inlet and the liquid inlet (and also the diffusion chamber in the embodiment in which it is provided), the mixing chamber being arranged to induce turbulence into the fluid flowing therethrough. The mixing chamber produces turbulent flow of the liquid and the oxygen flowing through the mixing chamber which acts to break-up the oxygen into small bubbles within the liquid so that they are more easily dissolved into the liquid in the mixing chamber and downstream in the apparatus, e.g. in the venturi. The mixing chamber may be provided in any suitable and desired way, i.e. to induce the necessary turbulent flow. For example, the mixing chamber may comprise one or more obstacles (e.g. barriers in the flow path) and/or a tortuous path.

If desirable, after passing through the apparatus and being oxygenated, some of the oxygenated liquid may be recycled, e.g. the apparatus may comprise a conduit arranged to recycle a portion of the oxygenated fluid from the outlet to the liquid inlet. Thus in one embodiment the conduit has one end in fluid communication with, and downstream of, the outlet, and another end in fluid communication with and upstream of the liquid inlet. Recycling some of the oxygenated liquid may help to increase the concentration of dissolved oxygen in the liquid owing to at least some of the liquid passing multiple times through the apparatus. In one embodiment, however, the apparatus is arranged to operate in a single pass production mode, i.e. with no recycling of the oxygenated liquid.

The oxygen may be supplied into the apparatus in any suitable and desired way. It may be supplied into the apparatus in a liquid and/or a gaseous form. In one embodiment the apparatus comprises a pressurised oxygen supply, e.g. a pressurised gas cylinder containing oxygen, in fluid communication with the oxygen inlet.

The flow rate of the liquid through the apparatus may be any suitable and desired value or range of values, e.g. depending on the viscosity of the liquid. In one embodiment the apparatus is arranged to deliver a flow rate of oxygenated liquid of between 0.01 ml/min and 100 l/min from the outlet of the apparatus, e.g. between 0.1 ml/min and 50 l/min, e.g. between 1 ml/min and 20 l/min, e.g. between 5 ml/min and 5 l/min.

The pressure of the liquid flowing through the apparatus may be any suitable and desired value or range of values. In one embodiment the apparatus is arranged to operate at a fluid pressure of between 0.1 and 5 bar, e.g. between 0.5 and 4 bar, e.g. approximately 3 bar.

Any of the apparatus and methods herein described may be used with any liquid as is suitable and desired. In this context, the term “liquid” thus includes not only liquids in the conventional sense but also materials which are flowable, e.g. a thickened or viscous liquid, or a flowable gel. Typically, the liquid for oxygenation will be water or a physiological salt solution.

The methods and apparatus herein described are capable of producing oxygenated solutions with a concentration of dissolved oxygen of greater than 20 mg/l, e.g. greater than 30 mg/L, e.g. greater than 40 mg/L, e.g. greater than 50 mg/L, e.g. greater than 60 mg/L, e.g. approximately 70 mg/L. Oxygenation levels up to about 100 mg/L, e.g. up to about 90 mg/L or up to 80 mg/L, may be achieved.

As will be appreciated, the concentration of dissolved oxygen able to be achieved depends on the temperature of the liquid flowing through the apparatus, with the achievable concentration generally increasing with decreasing temperature. Suitable temperatures for any of the oxygenation processes herein described may readily be selected by those skilled in the art.

In another set of embodiments, the antimicrobial compositions herein described may be prepared by oxygenation of an aqueous dispersion of the chemically modified cellulose nanofibrils. For example, these may be oxygenated using any of the apparatus and methods described in WO 02/26367, WO 2010/077962 and WO 2016/071691. In particular, they may be oxygenated using the method and apparatus described in WO 2016/071691. The OXY BIO System (Oxy Solutions, Oslo, Norway) may be used to oxygenate an aqueous dispersion of the charged cellulose nanofibrils as herein described.

The viscosity of an aqueous dispersion of the chemically modified cellulose nanofibrils will be dependent, at least in part, on the concentration of the nanocellulose. At lower concentrations (e.g. up to about 0.4 wt. %) these will be liquid, or thickened liquids, whereas at higher concentrations (e.g. above about 0.4 wt. %) these will be considered a “gel”. Any of the apparatus and methods described in WO 02/26367, WO 2010/077962 and WO 2016/071691 may be used to oxygenate liquids or flowable gels. The methods and apparatus described above for use in preparing an oxygenated solution may thus also be used to oxygenate an aqueous dispersion of the charged cellulose nanofibrils.

Thus, in one set of embodiments, the antimicrobial compositions herein described may be prepared by oxygenation of a dispersion of charged cellulose nanofibrils in an aqueous solution. For example, these may be produced by a method comprising the following steps:
introducing a liquid comprising an aqueous dispersion of charged cellulose nanofibrils as herein described into a piping network to form a flow stream;
injecting gaseous oxygen into the flow stream to produce a mixture of said liquid and oxygen bubbles; and
passing the flowing mixture of liquid and gaseous oxygen bubbles through a venturi which is arranged to dissolve the gas into the liquid passing through the venturi.

In this method, the term “liquid” encompasses liquids in the conventional sense and any aqueous materials which are flowable, e.g. a thickened or viscous liquid, or a flowable gel.

In this method, the liquid introduced into the piping network to form the flow stream may be pressurised, but it need not be. Suitable flow rates may be readily selected. In some embodiments, liquid flow rates may range from 1 L/min to 25 L/min. In certain embodiments, suitable oxygen flow rates may range from 0.1 L/min to 2.0 L/min. In cases where the liquid is pressurised at the point of introduction into the piping network, this may be pressurised to a pressure of from 1 to 5 bar.

The apparatus herein described is capable of producing an oxygenated composition with a concentration of dissolved oxygen of greater than 20 mg/l, e.g. greater than 30 mg/L, e.g. greater than 40 mg/L, e.g. greater than 50 mg/L, e.g. greater than 60 mg/L, e.g, approximately 70 mg/L. Oxygenation levels up to about 100 mg/L, e.g. up to about 90 mg/L or up to 80 mg/L, may be achieved.

If desirable, the viscosity of any oxygenated composition described herein may be increased by subjecting it to additional post-treatment steps. It may, for example, be desirable to increase the viscosity to transform a liquid composition to a more viscous liquid or to a hydrogel.

In one embodiment, the viscosity of a liquid nanocellulose composition as herein described (a “first nanocellulose composition”) may be increased by admixing with a second nanocellulose composition having a higher concentration of dispersed CNFs. The second nanocellulose composition may or may not be oxygenated. It may, for example, be non-oxygenated. As will be understood, the resulting composition will have a dissolved oxygen content of at least 20 mg/l. The components may be mixed in the desired amounts under controlled temperature conditions. Mixing at low temperatures (e.g. in the range 2 to 25° C., preferably at about 4 to 5° C.) and, preferably, under controlled pressure conditions is generally advisable to minimise the loss of oxygen. Stirring of the composition during preparation should also be controlled, e.g, minimised, to avoid the loss of oxygen. This preparation method is illustrated in Example 10 in which an oxygenated CNF composition containing 0.2 wt. % is mixed with a non-oxygenated CNF composition having a concentration of 0.4 wt. % whereby to increase its viscosity. As seen in this example, this can be done with minimum impact on the dissolved oxygen content.

Alternatively, oxygenated nanocellulose compositions having a higher viscosity may be prepared by mixing a highly viscous aqueous dispersion of the chemically modified cellulose nanofibrils (e.g. a hydrogel) with an aqueous solution (e.g. water or a saline solution) having the desired content of dissolved oxygen. Mixing of these components is effective to dissolve the viscous dispersion (e.g. hydrogel) and form a homogenous solution. To minimise the loss of oxygen, mixing should be carried out with minimum shear force. The aqueous solution having the desired oxygen content may be prepared using any of the apparatus and oxygenation methods herein described.

In another embodiment, the viscosity of a liquid nanocellulose composition as herein described may be increased by cross-linking of the charged nanofibrils. For example, cross-linking may be effected using divalent cations which are able to cross-link the nanofibrils through the —COO⁻ groups. Suitable divalent cations include, but are not limited to, Ca²⁺, Cu²⁺, Sr²⁺ and Ba²⁺. CaCl₂ may, for example, be used to cross-link the nanofibrils via Ca²⁺ cations. Suitable concentrations of cross-linking agents may readily be determined according to need, but may for example range from about 50 mM to about 100 mM.

An antimicrobial composition in the form of a hydrogel may alternatively be prepared by re-hydrating an aerogel which contains the charged cellulose nanofibrils using an oxygenated liquid which contains the required level of dissolved oxygen. Aerogels can be prepared by known methods. For example, these may be produced by freezing a hydrogel, e.g. at −20° C. and lyophilizing for a period of up to 24 hours using a Telstar LyoQuest −83 apparatus. The freezing temperature can be adjusted in order to modify the pore size of the aerogel. For example, this may be lowered to about −80° C. Suitable aerogels can be prepared by freezing and lyophilising a 3D printed hydrogel.

In one embodiment, the invention thus provides a method for the preparation of an antimicrobial composition as herein described, said method comprising the following steps: (i) preparing an aerogel comprising charged cellulose nanofibrils; and (ii) saturating said aerogel with an oxygenated liquid (e.g. oxygenated water or oxygenated saline) having a dissolved oxygen content of at least 20 mg/l whereby to form a hydrogel.

The antimicrobial properties of the compositions herein described make these suitable for medical use, for example in treating wounds. In a further aspect the invention thus provides a composition as herein described for use as an antimicrobial agent, for example for use in inhibiting the growth of at least one wound pathogen.

In a further aspect the invention provides the use of an antimicrobial composition as herein described in the manufacture of a medicament for use in a method for treating a wound.

In another aspect the invention provides a method for treating a wound, said method comprising the step of applying an effective amount of an antimicrobial composition as herein described to said wound. Optionally, said method may further comprise the step of applying a wound covering (herein referred to as a “secondary dressing”) following application of said antimicrobial composition.

In the treatment of a wound it may be beneficial to deliver other active agents to the wound site. In one embodiment, at least one other active substance may also be present in the composition, for example a combination of other active substances. These include substances known to be suitable for the treatment of wounds.

Other active agents which may be present in any of the compositions herein described include antibacterial agents, antifungal agents, antiviral agents, antibiotics, growth factors, cytokines, chemokines (e.g. macrophage chemo-attractant protein (MCP-1 or CCL2), nucleic acids, including DNA, RNA, siRNA, micro RNA, vitamins (e.g. vitamins A, C, E, B), minerals (e.g. zinc, copper, magnesium, iron, silver, gold), anaesthetics (e.g. benzocaine, lidocaine, pramoxine, dibucaine, prilocaine, phenol, hydrocortisone), anti-inflammatory agents (e.g.

corticosteroids, iodide solutions), moisturizers (e.g. hyaluronic acid, urea, lactic acid, lactate and glycolic acid), extracellular matrix proteins (e.g. collagen, hyaluronan, and elastin), enzymes (e.g, enzymes in the hatching fluid from fish roe, or in roe extracts such as salmon egg extract), stem cells from plants, extracts from eggs and eggshells (e.g. from salmon and hen's eggs), botanical extracts, fatty acids (e.g. omega-6 and omega-3 fatty acids, in particular polyunsaturated fatty acids), and skin penetration enhancers.

Growth factors exert potent and critical influence on normal wound healing. Wound repair is controlled by growth factors (platelet-derived growth factor [PDGF], keratinocyte growth factor, and transforming growth factor-β). PDGF is important for most phases of wound healing. Recombinant human variants of PDGF-BB (Becaplermin) have been successfully applied in diabetic and pressure ulcers. Growth factors which may be provided in the compositions include epidermal growth factor (EGF), platelet derived growth factor (PDGF), fibroblast growth factor (FGF), keratinocyte growth factor (KGF or FGF 7), vascular endothelial growth factor (VEGF), transforming growth factor (TGF-b1), insulin-like growth factor (IGF-1), human growth hormone and granulocyte-macrophage colony stimulating factor (GM-CSF).

Cytokines, e.g. the interleukin (IL) family and tumor necrosis factor-α family promote healing by various pathways, such as stimulating the production of components of the basement membrane, preventing dehydration, increasing inflammation and the formation of granulation tissue. IL-6 is produced by neutrophils and monocytes and has been shown to be important in initiating the healing response. It has a mitogenic and proliferative effect on keratinocytes and is chemoattractive to neutrophils. Examples of cytokines which may be present include the interleukin (IL) family, and tumor necrosis factor-α family.

Vitamins C (L-ascorbic acid), A (retinol), and E (tocopherol) show potent anti-oxidant and anti-inflammatory effects. Vitamin C deficiencies result in impaired healing, and have been linked to decreased collagen synthesis and fibroblast proliferation, decreased angiogenesis, increased capillary fragility, impaired immune response and increased susceptibility to wound infection. Similarly, vitamin A deficiency leads to impaired wound healing. The biological properties of vitamin A include anti-oxidant activity, increased fibroblast proliferation, modulation of cellular differentiation and proliferation, increased collagen and hyaluronate synthesis, and decreased MMP-mediated extracellular matrix degradation.

Several minerals have been shown to be important for optimal wound repair. Magnesium functions as a co-factor for many enzymes involved in protein and collagen synthesis, while copper is a required co-factor for cytochrome oxidase, for cytosolic anti-oxidant superoxide dismutase, and for the optimal cross-linking of collagen. Zinc is a co-factor for both RNA and DNA polymerase, and a zinc deficiency causes a significant impairment in wound healing. Iron is required for the hydroxylation of proline and lysine, and, as a result, severe iron deficiency can result in impaired collagen production.

Collagen plays a vital role in the natural wound healing process from the induction of clotting to the formation and final appearance of the final scar. It stimulates formation of fibroblasts and accelerates the migration of endothelial cells upon contact with damaged tissue. Chitosan accelerates granulation during the proliferative stage and wound healing.

Examples of anti-bacterial agents that may be present in the compositions include, but are not limited to, the following: alcohols, chlorine, peroxides, aldehydes, triclosan, triclocarban, benzalkonium chloride, linezolid, quinupristin-dalfopristin, daptomycin, oritavancin and dalbavancin, quinolones, and moxifloxacin.

The amount of any other active substances which may be present in the compositions according to the invention may readily be determined by those skilled in the art depending on the choice of active substance. Typically, this may be present in the range from 1 to 10 wt. %, e.g. 1 to 5 wt. % (based on the total weight of the composition).

In one embodiment, the compositions herein described may be substantially free from (e.g. free from) other active substances. For example, they need not include any additional antibacterial agents.

The compositions herein described are aqueous, but need not be purely aqueous. The compositions may comprise up to 99.8 wt. % water. Typically, these will comprise at least 50 wt. % water, more preferably at least 60 wt. % water, yet more preferably at least 70 wt. % water, e.g. at least 80 wt. % water. For example, the compositions herein described may contain from 95 to 99.8 wt. % water. A relatively high water content ensures a high oxygen level and thus may lead to rapid absorption of the dissolved oxygen into the skin.

The compositions according to the invention may comprise other optional components, e.g. components which maintain a buffered pH, or those which maintain osmolality in a range suitable for the intended application, or which maintain stability of the composition. Other components which may be present thus include buffers, pH adjusting agents, osmolality adjusting agents, preservatives (e.g. anti-microbial agents), anti-oxidants, fragrances, coloring agents, etc.

The presence of a buffer serves to adjust the pH to physiological levels, e.g. in the range from 3 to 9, preferably from 4 to 7, e.g. about 5.5. A suitable choice of buffer can also aid in controlling the ionic strength of the compositions. Examples of buffers which may be employed include citrate, phosphate, carbonate, and acetate.

Isotonic aqueous buffers, such as phosphate, are particularly preferred. Examples of suitable buffers include TRIS, PBS, HEPES.

Wounds with an alkaline pH have lower healing rates than those with a pH closer to neutral. Some studies have also shown that an acidic environment in the wound supports the natural healing process and controls microbial infections. Chronic wounds typically have an elevated alkaline environment and may, for example, have a pH in the range of 7.15 to 8.9. In the treatment of wounds, especially chronic wounds, an acidic pH may thus be advantageous. In one embodiment, the compositions may therefore be buffered to have a pH in the range from 2 to 7. For example, these may be buffered to a pH in the range from 3 to 6.5, preferably from 5 to 6, more preferably from 5 to 5.5, e.g. about 5.1 to about 5.5. pH adjusting agents which may be present include sodium hydroxide, hydrochloric acid, acetic acid, boric acid, ascorbic acid, hyaluronic acid, and citric acid.

Salts may also be present in order to adjust the osmolality of the compositions and thus enhance their tolerability in vivo. Any suitable salt known in the art for adjusting osmolality may be employed. Osmolality may be adjusted depending on the nature of the wound. For example those with excessive exudate may benefit from a hypertonic composition, whereas for others a hypotonic or isotonic composition may be more appropriate. One example of a suitable salt is sodium chloride. This may be added in an amount ranging from about 0.05 to about 2 wt. %, e.g. about 0.2 to about 1 wt. % (based on the total weight of the composition) to form an isotonic composition. Higher or lower amounts may be added as required to obtain a hypotonic or hypertonic composition. Where the composition is a hydrogel, the presence of sodium chloride may further serve to strengthen the gel, and to increase its bioadhesive force.

Where the composition is in the form of a hydrogel, the choice of any additional components should take into account any negative impact it may have on the strength of the gel. Agents which may reduce the strength of the gel should thus either be used sparingly or not at all.

Suitable preservatives which may be present in the compositions include, but are not limited to, benzalkonium chloride, sodium chloride, parabens, vitamin E, disodium EDTA, glycerin, and ethanol.

The presence of one or more antioxidants may serve to extend the shelf life of the compositions herein described, for example where these may contain any other components which are sensitive to oxidation. Examples of suitable antioxidants which may be present include ascorbic acid and ascorbic acid salts (e.g. sodium ascorbate, potassium ascorbate and calcium ascorbate); fatty acid esters of ascorbic acid such as ascorbyl palmitate and ascorbyl stearate; tocopherols such as alpha-tocopherol, gamma-tocopherol and delta-tocopherol; propyl gallate, octyl gallate, dodecyl gallate or ethyl gallate; guaiac resin; erythorbic acid, sodium erythorbate, erythorbin acid or sodium erthorbin; tert-butylquinone (TBHQ); butylated hydroxyanisole (BHA); butylated hydroxytoluene (BHT); anoxomer and ethoxyquin. Preferred for use in the invention are those antioxidants which are water-soluble such as, for example, ascorbic acid and ascorbate salts.

The optimum amount of antioxidant(s) in the compositions of the invention will depend on a number of factors including the oxygen level of the composition, the presence and amount of any oxygen-sensitive compounds in the composition, etc. Suitable levels may readily be determined by those skilled in the art. However, the level of antioxidant will typically be at least 0.001 wt. %, especially at least 0.01 or at least 0.03 wt. %. The level of antioxidant will typically be less than 5 wt. %, especially less than 2 or 1 wt. %, e.g. between 0.02 and 0.5 wt. % or between 0.05 and 0.2 wt. %.

Skin penetration enhancers may also be present and these may have a beneficial effect in enhancing the activity of the compositions. Any of the skin penetration enhancing agents known and described in the pharmaceutical literature may be used. These may include, but are not limited to, any of the following: fatty acids (e.g. oleic acid), dialkyl sulphoxides (such as dimethylsulphoxide, DMSO), Azones (e.g. laurocapram), pyrrolidones and derivatives (e.g. 2-pyrrolidone, 2P), alcohols and alkanols (e.g. ethanol, decanol, isopropanol), glycols (e.g. propylene glycol), and surfactants (e.g. dodecyl sulphate). Examples of other skin penetration enhancing agents include propylene glycol laurate, propylene glycol monolaurate, propylene glycol monocaprylate, isopropyl myristate, sodium lauryl sulphate, dodecyl pyridinium chloride, oleic acid, propylene glycol, diethylene glycol monoethyl ether, nicotinic acid esters, hydrogenated soya phospholipids, essential oils, alcohols (such as ethanol, isopropanol, n-octanol and decanol) terpenes, N methyl-2-pyrrolidine, polyethylene glycol succinate (TPGS), Tween 80 and other surfactants, and dimethyl-beta-cyclodextrin. Where present, any surface penetration enhancing agents may be provided in an amount in the range of from 0.1 to 10 wt. %, e.g. about 5 wt. %.

In an embodiment, the compositions according to the invention consist essentially of water, dissolved oxygen, charged cellulose nanofibrils, and optionally one or more pharmaceutically acceptable carriers or excipients. As used herein, the term “consisting essentially of” means that the compositions do not comprise any other components which materially affect their properties when in use, such as other pharmaceutically acceptable agents which may typically be used in wound treatment.

Where the compositions according to the invention contain any of the other components herein described, these may be incorporated into the oxygenated cellulose-containing composition or into any components of the composition, for example an oxygenated liquid to be used in their preparation. These may be added with simple mixing of the components in the desired amounts under controlled temperature conditions, for example at low temperatures (e.g. in the range 2 to 25° C., preferably 4 to 5° C.) and, preferably, under controlled pressure conditions to minimise the loss of oxygen. Stirring or agitation of the compositions during preparation should be controlled, e.g. minimised, to avoid the loss of oxygen. In a preferred embodiment, other components may be added to the composition prior to oxygenation to avoid the need to mix or stir the composition once oxygenated.

For use in vivo the compositions herein described should be sterilised. This can be achieved by methods known in the art. The conditions for sterilisation should be selected such that the product maintains its desired antimicrobial properties whilst minimising the level of viable microorganisms in the product during storage. In some cases, the separate components of the compositions may be sterilized prior to mixing. Sterilization of the cellulose nanofibrils may, for example, be achieved by electron beam radiation or gamma radiation. Alternatively, the final compositions may be sterilized once oxygenated. In this case, sterilization may similarly be achieved by gamma or electron beam irradiation or by other means such as microfiltration using a filter having a small pore size (e.g. about 0.22 μm). The ability to filter the composition will be dependent on its final viscosity, but when cooled sufficiently such that this is in a liquid state microfiltration will generally be feasible.

The compositions herein described may be incorporated into a wound covering, for example these may be provided in, or as a component of, a conventional dressing, bandage or any other suitable wound covering. In another aspect, the invention thus provides a wound covering having incorporated therein an antimicrobial composition as herein described. In use, the wound covering may be applied to the target tissues (e.g. the surface of the skin) such that the antimicrobial composition contained therein comes into contact with the underlying body tissues.

In one embodiment, the compositions may be incorporated into a bandage, gauze, patch or absorptive pad, or a portion thereof, and packaged ready for use. For example, a liquid composition may be soaked into a suitable wound covering (e.g. an absorbent pad) and packaged ready for use. The bandage, gauze, patch or absorptive pad may be packaged under a vacuum or pressure. Alternatively, the wound covering containing the composition may be prepared at the point of use by application of the composition to a suitable wound covering (e.g. by soaking or immersion of the wound covering in any liquid composition) immediately prior to application to the body tissues. In another aspect the invention thus provides a kit for use in treating a wound, the kit comprising: (a) a sterilised, sealed container or package containing an antimicrobial composition as herein described; and (b) a wound covering, e.g. a wound dressing, bandage, gauze, patch or absorptive pad. The kit may additionally comprise printed instructions for use of the components of the kit in the treatment of a wound.

In one embodiment, the compositions herein described may be provided in the form of a hydrogel which can be used as a wound dressing. In another aspect, the invention thus provides a wound dressing in the form of a hydrogel comprising charged cellulose nanofibrils, wherein said hydrogel has a dissolved oxygen content of at least 20 mg/l.

When used as a wound dressing, the hydrogel can be provided in any desired shape or size suitable for application to the wound site. For example, it may be provided as a flexible structure or “construct” (e.g. a sheet) of hydrogel material. Such constructs may be produced by three-dimensional (3D) printing of an oxygenated cellulose material as described herein. Methods for 3D printing of hydrogel materials are well known in the art and may be performed using any conventional 3D printing apparatus such as a Regemat3D printing unit. 3D printed structures may be single or multi-layered depending on their intended use, for example the nature and extent of the wound to be treated. Once “printed”, the hydrogel constructs may be subjected to cross-linking to increase their viscosity and enhance their mechanical properties, e.g. to provide a self-holding, yet flexible, 3D-structure. Cross-linking may be effected using any of the cross-linking agents herein described, for example by immersing the 3D printed hydrogel construct in a solution of the selected cross-linking agent. Immersion in a solution of CaCl₂ for several hours, e.g. up to 24 hours, may be suitable.

In use, the hydrogel dressing may be applied directly to the wound site. If required, it may be cut to size at the point of use.

The compositions herein described may be packaged in a suitable, sealed container or packaging which is sterilised, e.g. by steam sterilization (i.e. autoclaving) or gamma irradiation. Autoclaving may be carried out at a temperature in the range from 105 to 150° C., preferably 120 to 135° C. for a period of time which is sufficient to kill microorganisms. Sterilization times are dependent on the type of item to be sterilized, e.g. metal, plastic, etc., but can be expected to be in the range of from 1 to 60 minutes, e.g. 4 to 45 minutes. Typical steam sterilizing temperatures may be 121° C. or 132° C.

Suitable types of containers may be selected according to the nature of the hydrogel, and its intended use, e.g. the type of wound to be treated, the duration of treatment and whether multiple uses are envisaged. Suitable packaging includes vials, loaded syringes, tubes, pouches, bottles, etc. In each case these should be effectively sealed in order to avoid depletion of oxygen on storage. Vials may, for example, be provided with a suitable twist to break cap.

Packages may be intended for single or multiple use. Where these are intended for multiple use it is important that the remaining content of the package can be sealed after opening and following the delivery of each dose of composition in order to maintain the sterility of the product and minimise the loss of oxygen. Containers having a one-way pump may be suitable. Alternatively, the compositions may be provided in individual doses, e.g. in sachets, small tubes or bottles which contain an amount sufficient for a single application to the skin. Single use ampoules are preferred.

Maintaining high and stable oxygen levels in the compositions when stored is essential. Suitable storage containers, lids and the materials used for their preparation should be chosen accordingly. These should have low susceptibility to penetration of gases, especially oxygen. Preferably these should be impermeable to gases. Suitable containers include glass jars, vials and tubes, and disposable plastic containers such as those made from polyethylene terephthalate (PET) or its copolymers. Optionally any plastic containers (e.g. those made from PET or its copolymers) may comprise additional components to enhance their gas barrier properties. Such materials are, for example, described in US 2007/0082156 and WO 2010/068606, the contents of which are incorporated herein by reference.

Ideally, any storage containers should have minimum oxygen permeability in order to maximise shelf life of the product. Typically a suitable shelf life is a minimum of about 6 months, preferably 6 to 12 months under ambient conditions. Shelf life may be extended by storage at lower temperatures, e.g. under refrigeration at a temperature in the range from 2 to 4° C. During storage for the intended shelf life, it is preferable that the oxygen content of the product should not be reduced by more than 25%.

The compositions herein described may be applied to any wound site where delivery of oxygen is desirable. The method of delivery will be dependent on the form of the product, i.e. whether this is used as a liquid (e.g. a thickened or viscous liquid) or a gel, or whether this is provided as a component in a wound covering as herein described. For any therapeutic use, in order to maintain the sterility of the product it is generally envisaged that these should be applied by sterile means. For example, these may be applied to the target area using an applicator (e.g. from a syringe).

Wounds typically involve interruption in the integrity of the skin. When skin is damaged or removed, e.g. removed by surgery, burned, lacerated or abraided, its protective function is lost. All types of skin wound may be treated in accordance with the invention, including both acute and chronic wounds.

Acute wounds are usually tissue injuries that heal completely with minimal scarring within the expected timeframe, e.g. up to 10 days. Primary causes of acute wounds include mechanical injuries due to external factors such as abrasions and tears which are caused by frictional contact between the skin and hard surfaces. Mechanical injuries also include penetrating wounds caused by knives and surgical wounds caused by surgical incision (e.g. in the removal of tumors). Acute wounds also include burns and chemical injuries, such as those which may arise from radiation, electricity, corrosive chemicals and thermal sources (both hot and cold). Burn wounds may be classified according to their severity, e.g. as first, second or third degree burns.

Chronic wounds arise from tissue injuries that heal slowly, e.g. injuries that have not healed after about 12 weeks, and often recur. Such wounds typically fail to heal due to repeated tissue injury or underlying physiological conditions such as diabetes, obesity, malignancies, persistent infections, poor primary treatment and other patient-related factors. Chronic wounds include skin ulcers, such as decubitis ulcers (e.g. bedsores or pressure sores), leg ulcers (whether venous, arterial, ischaemic or traumatic in origin), and diabetic ulcers. Venous leg ulcers are caused by venous insufficiency due to malfunctioning of the valves in the veins in the leg and may lead to pulmonary embolia which is a life-threatening condition. They are costly to treat, often requiring hospitalization. Arterial leg ulcers are caused by poor functioning or occlusion of the arteries in the leg and may arise from conditions such as arteriosclerosis. Diabetic ulcers arise from impaired microcirculation as a result of diabetes. In the case of diabetic ulcers, failure to heal can often lead to loss of a limb.

Wounds may also be classified according to the number of skin layers and area of skin which is affected. In a superficial wound the injury affects the epidermal skin surface alone. Injury involving both the epidermis and the deeper dermal layers, including the blood vessels, sweat glands and hair follicles, may be referred to as a partial thickness wound. A full thickness wound occurs when the underlying subcutaneous fat or deeper tissues are damaged in addition to the epidermis and dermal layers.

When used in the treatment of wounds, the compositions of the invention increase the rate of wound healing through improved oxygenation, whilst simultaneously retaining moisture at the wound site and protecting against infection. Wounds and burns are particularly susceptible to infection where the tissue is destroyed or badly damaged, such as in second or third degree burns. In such cases, application of the compositions herein described can also prevent bacterial infection as well as act therapeutically to heal the damaged tissue.

The compositions herein described are particularly suitable for use in the treatment of wounds which are infected, for example chronic wounds. They may be used in the treatment of both aerobic and anaerobic bacterial and fungal infections of the skin due to the toxicity of oxygen to such pathogenic organisms. Fungal infections may be associated with enterococcus, enterobacteriacea, clostridium, B. fragilis, streptococcus, pyogenis. Examples of invasive fungal infections include those associated with mucorales, aspergilus.

Both aerobic and anaerobic bacteria may also be found in infected wounds and areas of skin burn. Anaerobic bacterial infections which may be treated using the compositions of the invention include Bacteroides species, and Clostridium species. Aerobic bacterial infections which may be treated using the compositions include Pseudomonas species (e.g. Pseudomonas aeruginosa), Enterococcus species, Enterobacteriacea species, Bacillus species, Streptococcus species, and Staphylococcus species (e.g. Staphylococcus aureus). The compositions are particularly suitable for use in treating wounds harbouring Pseudomonas and/or Staphylococcus species, e.g. Pseudomonas aeruginosa and/or Staphylococcus aureus.

In one embodiment, the compositions herein described may be used to prevent the formation of a bacterial biofilm and/or to treat a bacterial biofilm on a body surface. Treatment will typically involve disruption, removal or detachment of at least part of the biofilm from the body surface.

The subject to be treated may be any mammal. Although typically the subject will be a human, the methods herein described are equally suited to the treatment of non-human mammals. Veterinary use of the compositions is thus envisaged within the scope of the invention.

The compositions may be applied in a variety of different ways depending on factors such as the area to be treated, the nature of the condition, the subject to be treated, etc. These may be applied to any area of the body including the face, chest, arms, legs or hands. Typically, they will be applied to the skin. The method of application to the skin may be dependent on the viscosity of the composition but may include application by rubbing, soaking, immersion, continuous perfusion, injection, etc.

Dependent on their viscosity, the compositions may be applied by the fingers. However, in order to maintain sterility it is generally envisaged that these will be applied by sterile means, for example using an applicator. Applicators known for use in applying dermal products may be used depending on the nature of the formulation, especially its viscosity. For example, this may be applied with a spatula.

The compositions may be applied directly to the target tissue, i.e. the wound, and thus serve to form a “primary dressing”. Typically this will require a secondary dressing to protect the composition and to ensure that this remains in place for the duration of the treatment. The secondary dressing should be flexible and able to conform to the wound site. Typically this dressing will take the form of a sheet of conventional wound dressing material which may be cut to the appropriate size and shape depending on the area of tissue to be treated.

Any secondary dressing should ideally be of limited permeability to water and/or oxygen, e.g. this should be substantially impermeable to water and/or oxygen. The use of an occlusive dressing not only ensures that the dissolved oxygen present in the underlying composition is delivered to the skin, but it also serves to maintain a moist healing environment for the wound. By “substantially impermeable to oxygen” is meant that less than 25% of the oxygen content of the hydrogel may be lost through the dressing.

In dealing with repair and healing of a wound, it may be necessary to control exudate from the wound. This may involve drainage of exudate from the wound or absorption using a suitably absorbent dressing. Maintaining an optimal level of moisture at the site of the compromised tissue is also important, particularly in cases where there is heavy production of exudate. The use of a dressing helps to achieve this, In one embodiment the secondary dressing may thus be highly absorbent, particularly in the case of treating any wound with a high level of exudate. Examples of suitable dressings are known in the art and may readily be selected according to the type of wound, its size and location. Known dressings include both synthetic and biological dressings, such as synthetic films, alginates, hydrocolloids, hydrogels and collagen dressings. Those that are substantially impermeable to the passage of oxygen include polyesters and polyolefins. If desired the wound may also be covered with a compression bandage. This may be beneficial, for example, when treating venous ulcers.

In use, the composition is applied directly to the wound site or as close as possible to this. Preferably this should be in direct contact with the wound bed. A suitable secondary dressing is then applied over the composition and, if required, secured in place using tape, gauze or any other suitable means to secure this to intact skin. The secondary dressing may be temporary so that this may, if required, be removed and replaced with a fresh dressing. In one embodiment, the secondary dressing may be coated, in part, with an adhesive which is capable of securing this to the skin. For example, the dressing may have adhesive around its periphery. Suitable adhesive materials are known in the art and include, for example, polyisobutylene, polysilicone and polyacrylate. Where the dressing is supplied with an adhesive portion, this will generally also have a release liner, e.g. a siliconised polyester film, which is removed prior to use.

The duration of treatment will depend on the nature of the wound and the oxygen content of the composition applied to the skin. Typically, the dressing may be used on the wound for several days, e.g. up to 3 days. Use of the dressing for several days further reduces the cost of the treatment and reduces the trauma involved in changing of the dressing (e.g. where this may be required to be changed every day or several times a day). Delivery of oxygen from the dressing may be controlled. Controlled release relates to a release of oxygen over a predetermined period of time from 7 hours to 2 days. The delivery of oxygen is preferably substantially continuous during this period meaning the delivery is substantially uninterrupted.

In some cases, a further application of the composition may be desirable and this can be repeated as often as required. In order to change the dressing, the oxygen-depleted composition may easily be removed from the wound by gentle irrigation with a physiologically acceptable solution, such as sterile water or saline solution. Oxygenated water or oxygenated saline may also be used for this purpose. Irrigation of the wound between changing of the dressing also serves to cleanse the wound to remove dead or necrotic tissue.

Wound healing has several different phases which may not all be targeted by a particular hydrogel or dressing. Accordingly, the nature of the composition and any secondary dressing may be adjusted not only for different types of wound (e.g. acute, chronic, dry, exuding, etc.) but also for different stages in the healing of the wound. This includes, in particular, varying the oxygen content of the different compositions for the different stages of treatment. During the early stages of wound healing, low pO₂ (hypoxia) is an essential stimulator of growth factors, cytokines, gene activation and angiogenesis, whereas normal (normoxia) or increased (hyperoxia) levels of pO₂ are more favorable during the subsequent stages of wound healing. Fibroblast and endothelial cell proliferation, for instance, occurs best at a pO₂ of 30 to 80 mm Hg and collagen synthesis, neovascularization and epithelialization all require a pO₂ between 20 and 60 mm Hg.

Due to their ease of use, the wound treatments herein described may be used as home care, thereby reducing treatment costs and avoiding the need for hospitalization of patients. These also allow for full mobility for patients during treatment without the need for hospitalization, oxygen tanks or additional equipment. This increases the quality of life for patients.

The compositions herein described are intended for dermal use on the skin of a mammal, preferably a human subject. As such, these are compatible not only with the skin, but also with mucous membranes, nails and hair. Typically, these will also be non-irritant and well-tolerated when applied to the skin.

The invention will now be described further with reference to the following non-limiting Examples and the accompanying figures in which:

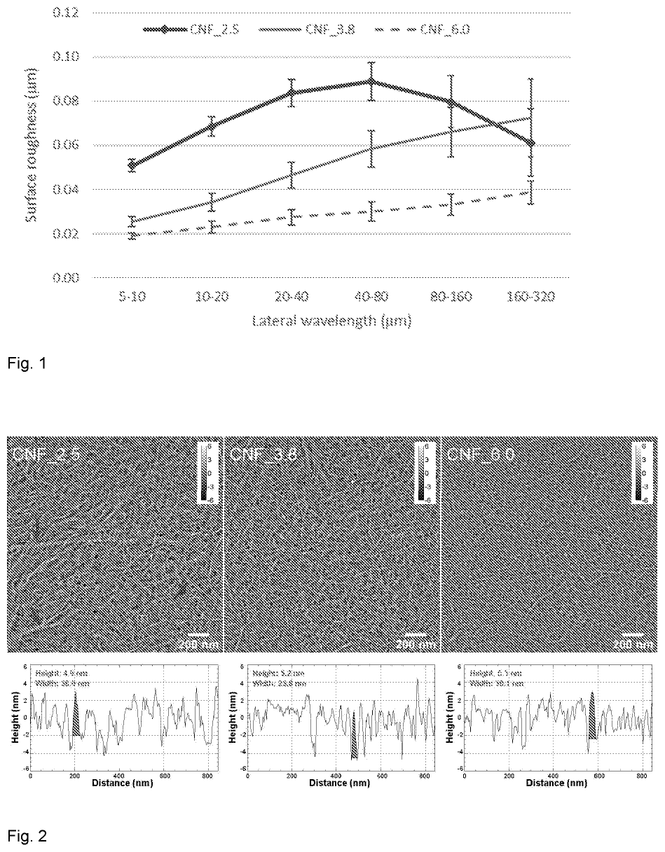

FIG. 1 shows the laser profilometry quantification of CNF film roughness in CNFs produced with increasing oxidation. The mean values for each lateral wavelength are given with the standard deviation of the mean (n=10).

FIG. 2 shows AFM analysis of samples CNF_2.5, CNF_3.8 and CNF_6.0. The relatively thicker nanofibrils in the CNF_2.5 sample are indicated by arrows. The height plots were acquired at the middle of each image, indicated by a dotted line. Calibration and scale bars are given in nanometers. Height and width is measured on a single nanofibril (colored black) from the profile plot.

FIG. 3 shows the Brookfield viscosity measured at various speeds for CNFs produced with increasing oxidation.

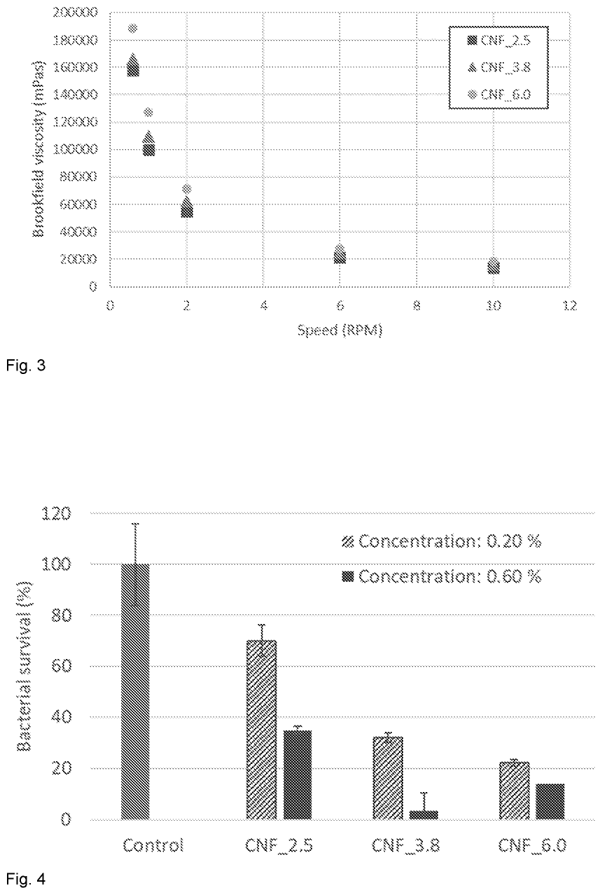

FIG. 4 shows the antimicrobial effect of CNF gels on P. aeruginosa after 24 hours exposure, correlated to the negative control BHI100, which is set to 100%. The bars represent average and error bars represents SEM. N=5 in all groups.

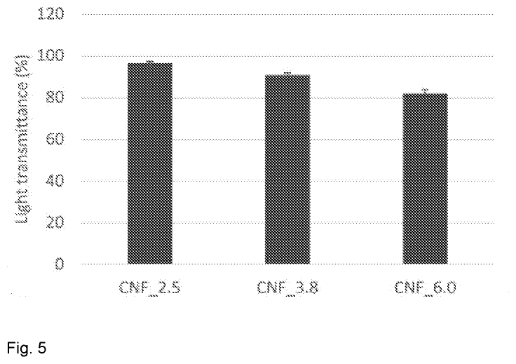

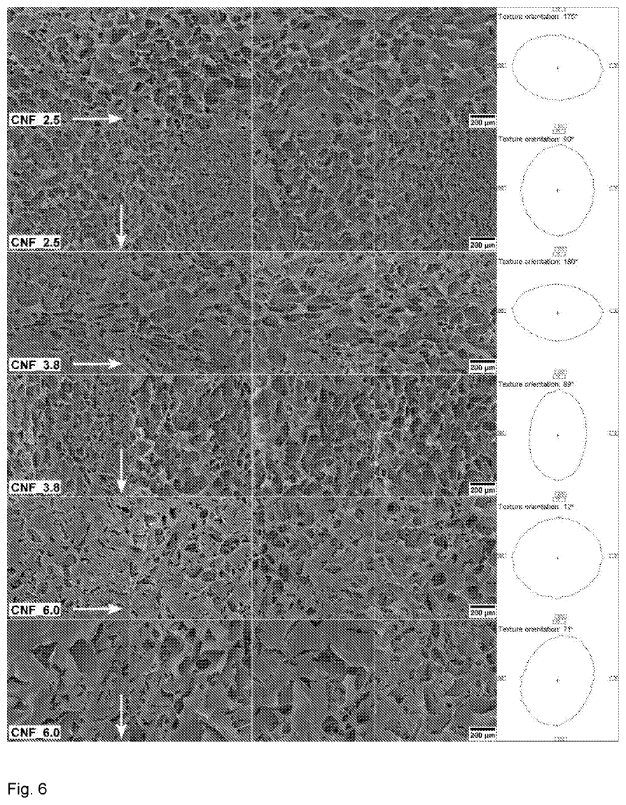

FIG. 5 shows the quantification of light transmittance of 3D printed constructs (the mean value is given with the standard deviation, n=4), Target dimensions of the 3D printed constructs were 20 mm×40 mm×2 mm.

FIG. 6 shows an SEM assessment of freeze-dried constructs. The four columns provide four replicate SEM images for each series. The arrows indicate the printing direction. The right column yields the polar plots showing the main orientation of the surface structure.

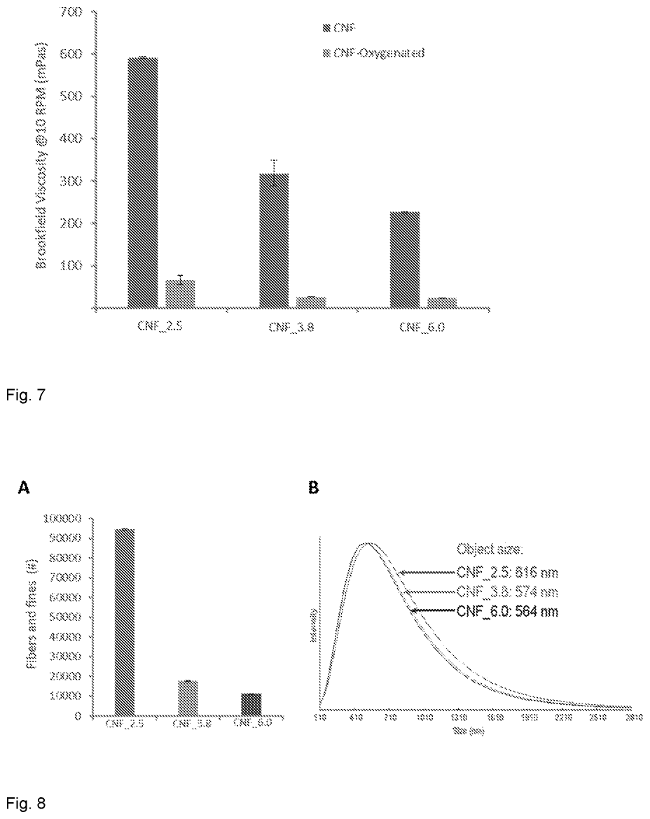

FIG. 7 shows the Brookfield viscosity of 0.2 wt. % CNF and oxygenated CNF for CNF_2.5, CNF_3.8 and CNF_6.0 (Table 1). Data are expressed as average±SEM (n=10).

FIG. 8 shows an assessment of CNF dispersions with (A) a FiberTester (residual fibres and fines), and (B) a nanoparticle analyser (nano-sized fibres).

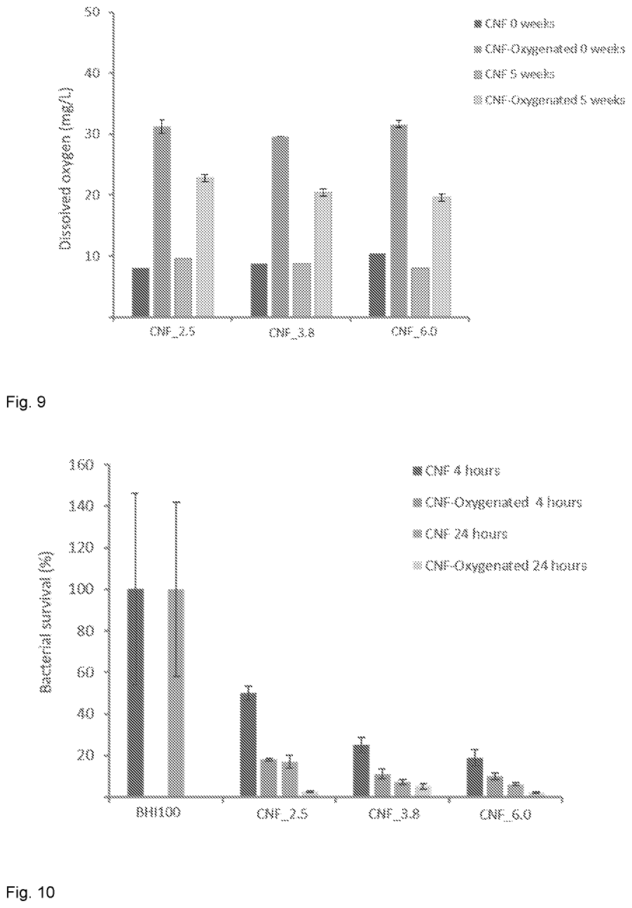

FIG. 9 shows the oxygenation of CNF and quantification of dissolved oxygen (DO). 0.2 wt. % CNF with different oxidation levels (CNF_2.5, CNF_3.8 and CNF_6.0, Table 1) was oxygenated and stored in sealed glass vials at room temperature (22° C.). The DO concentrations were measured at production date and 5 weeks later. Duplicate measurements for oxygenated CNF and singular measurements for CNF. Data are expressed as average±SEM.

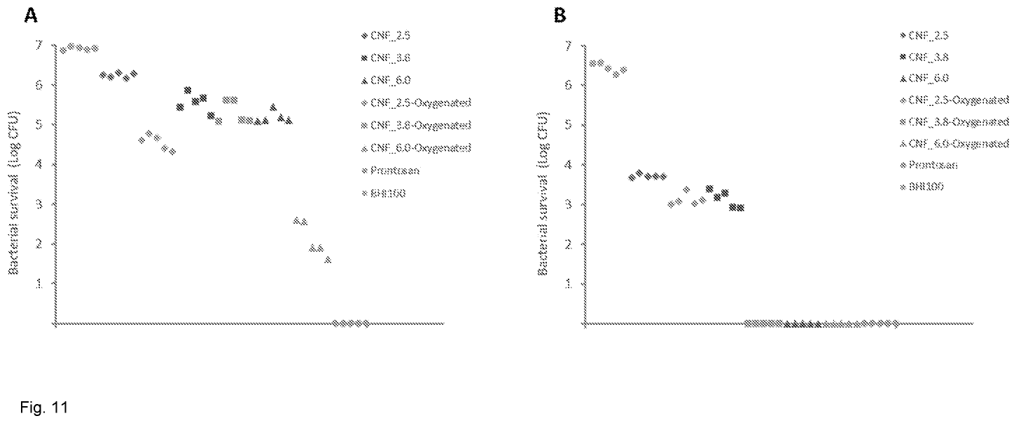

FIG. 10 shows the antimicrobial effect of CNF and oxygenated CNF on P. aeruginosa. Bacterial survival of 0.2 wt. % CNF with different oxidation levels (CNF_2.5, CNF_3.8 and CNF_6.0, Table 1) on P. aeruginosa after 4 or 24 hours. Data are expressed as average±SEM. n=5 in all groups, except for CNF_6.0 4 hours and CNF 24 hours (n=4). BHI100 was used as negative control.

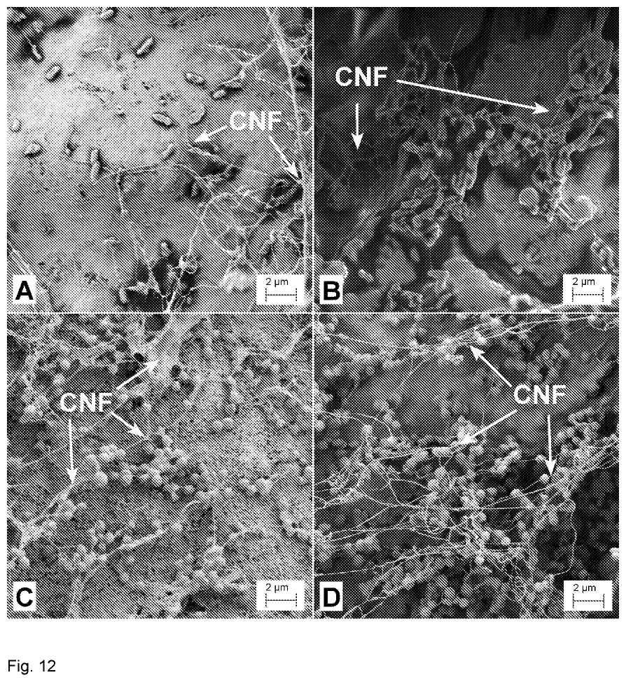

FIG. 11 shows the antimicrobial effect of CNF and oxygenated CNF on P. aeruginosa and S. aureus. Bacterial survival (Log10 CFU) of 0.2 wt. % CNF with different oxidation levels (CNF_2.5, CNF_3.8 and CNF_6.0, Table 1) on (A) P. aeruginosa and (B) S. aureus after 24 hours exposure. Data are expressed as Log10. N=5 in all groups, except CNF_3.8 in FIG. B (n=4). BHI100 and Prontosan were used as negative control and positive control, respectively.

FIG. 12 shows an SEM assessment of bacterial biofilms: (A) P. aeruginosa and CNF_6.0; (B) P. aeruginosa and CNF_6.0-Oxygenated; (C) S. aureus and CNF_6.0; and (D) S. aureus and CNF_6.0-Oxygenated.

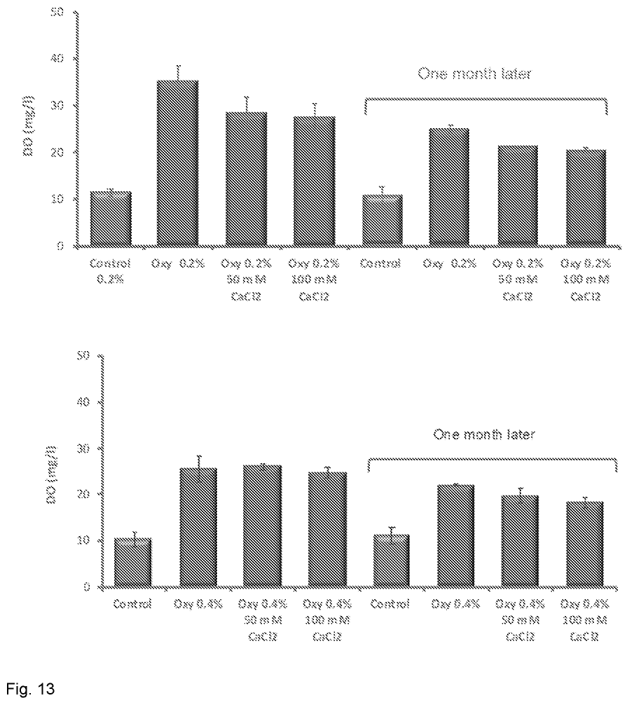

FIG. 13 shows the effect of cross-linking oxygenated CNF with CaCl₂. Upper figure: dissolved oxygen (DO) in 0.2 wt. % oxygenated CNFs with and without CaCl₂ (50 mM or 100 mM), N=3. Lower figure: dissolved oxygen (DO) in 0.4 wt. % oxygenated CNFs with and without CaCl₂ (50 mM or 100 mM), N=3 except for “Oxy 0.4% 100 mM CaCl₂” (N=1).

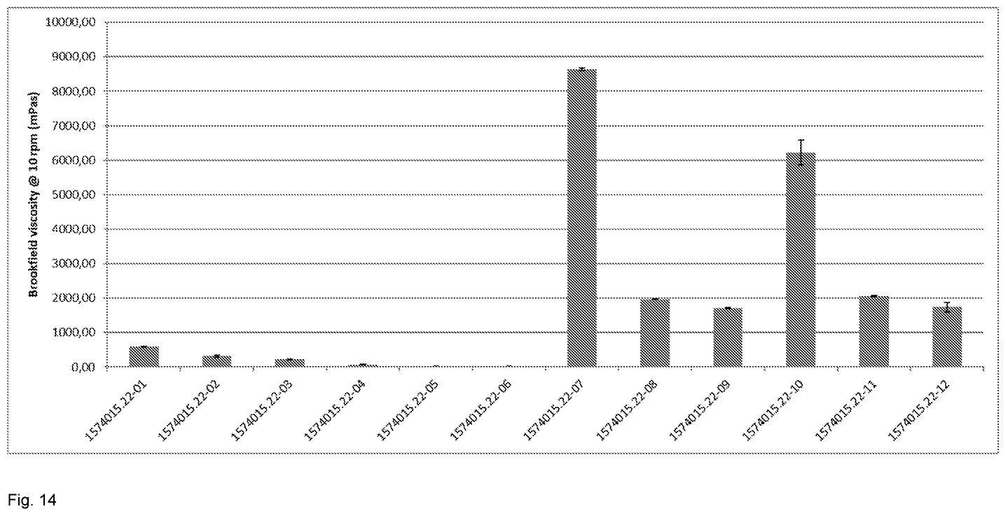

FIG. 14 shows the Brookfield viscosities of CNFs at 0.2 wt. % and 0.4 wt. % (measured at 10 RPM).

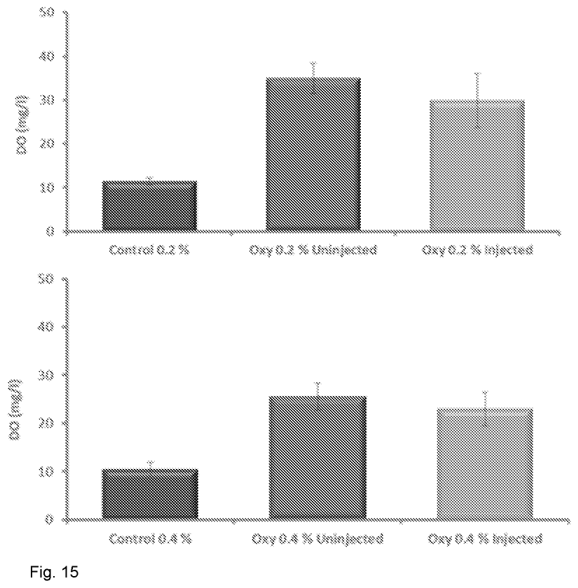

FIG. 15 shows the dissolved oxygen (DO) content of CNFs injected through a 50 ml needle tip with 18G cannula. Upper figure: 0.2 wt. % CNF. Lower figure: 0.4 wt. % CNF. N=3.

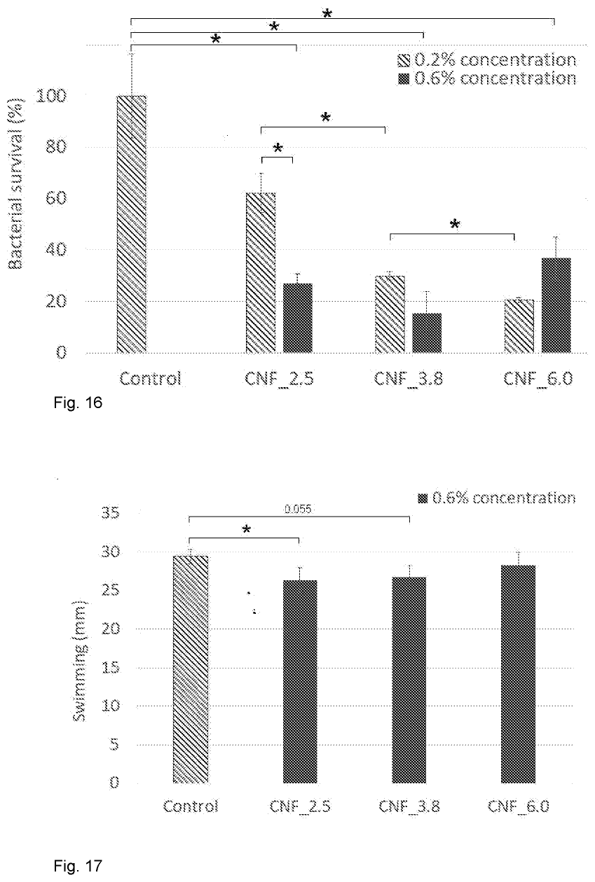

FIG. 16 shows the antibacterial effect of CNF gels on P. aeruginosa after 24 hours exposure, correlated to the negative control BHI100, which was set to 100%. The bars represent average and the error bars represent standard error of the mean. n=15 in all groups. All samples were significantly different compared to the control (*, p<0.05).

FIG. 17 shows swimming levels of P. aeruginosa in agar gels containing CNFs (0.6 wt. %). The bars represent average and the error bars represent standard error of the mean, n=3 in all groups (*, p<0.05).

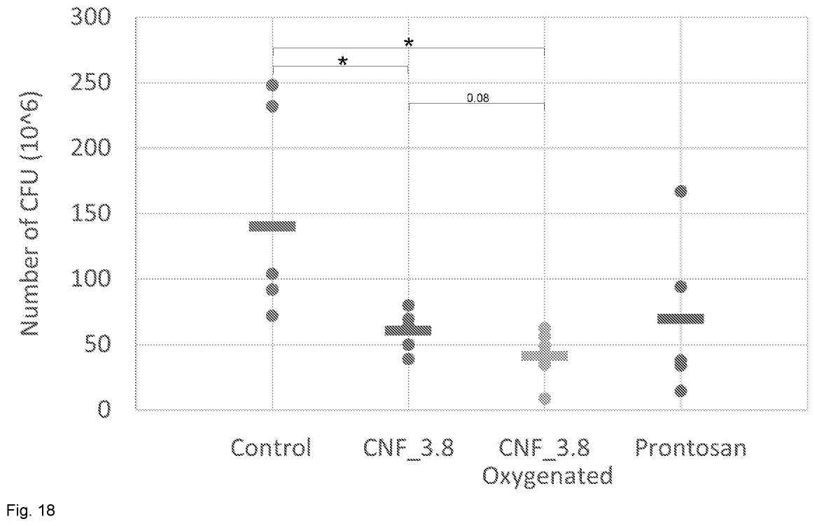

FIG. 18 shows the antimicrobial effect of CNF_3.8 and oxygenated CNF_3.8, assessed in vivo. Data are expressed as number of CFU. n=5 in all groups. (*) denotes significant difference (p<0.05).

### EXAMPLES

### Example 1—Preparation of Cellulose Nanofibrils (CNFs) and Characterisation

Preparation of CNFs:

Pinus radiata kraft pulp fibers were washed and autoclaved using NaOH as described by Nordli et al. (Carbohydrate Polymer 150, 65-73, 2016). This was performed to reduce the amount of endotoxins (Nordli et al., ACS Applied Bio Materials 2(3), 1107-1118, 2019). CNFs with varying surface chemistry were produced by TEMPO-mediated oxidation, applying three levels of oxidation, i.e. 2.5, 3.8 and 6.0 mmol hypochlorite (NaClO)/g cellulose and defined as CNF_2.5, CNF_3.8 and CNF_6.0, respectively (Saito et al., Biomacromolecules 5(5), 1983-1989, 2004). The CNFs were collected after passing the oxidized cellulose fibres three times through a homogenizer (Rannie 15 type 12.56X homogenizer, operated at 1000 bar pressure).

Characterisation of CA/Fs:

The content of carboxylic acid groups was quantified by conductometric titration according to Saito et al. (Biomacromolecules 5(5), 1983-1989, 2004). The content of aldehyde groups was determined based on a spectrophotometric method previously described by Jausovec et al. (Carbohydrate Polymer 116, 74-85, 2015).

The CNF gels (concentration 0.6 wt. %) were printed on microscopy slides using a Regemat3D printing unit (version 1.0, Regemat3D, Granada, Spain). Solid areas of 10×20 mm were printed, 2 layers, using a nozzle of 0.58 mm and flow 3 mm/s. The gels were allowed to dry at room temperature (23° C.) and 40% relative humidity. A layer of gold was deposited on the printed structures and 10 laser profilometry images (1×1 mm) were acquired with a resolution of 1 μm/pixel. The laser profilometry images were bandpass-filtered and the surface roughness (root-mean-square) was quantified at various lateral wavelengths (Chinga-Carrasco et al., Micron 56, 80-84, 2014).

Atomic force microscopy (AFM) was performed on the three CNF samples. The samples were analyzed with a Veeco multimode V at room temperature. The AFM tips had a spring constant ˜0.4 N m⁻¹ (Bruker AFM probes). The assessed local areas were 2×2 μm, with a resolution of 1.95 nm/pixel.

Viscosity of the CNFs was assessed with a Brookfield viscometer (Brookfield DV2TRV). The assessment was performed using spindle V-73 at a temperature of 23° C.±1° C. and at the following speeds: 0.6, 1, 2, 6 and 10 RPM.

Results and Discussion:

The carboxyl and aldehyde contents of the CNF gels and surface roughness of the CNF films are shown in Table 1:

TABLE 1

| | NaClO used | | | |
|---|---|---|---|---|
| | during | Carboxyl | Aldehyde | |
| | oxidation of | content of | content of | CNF film |
| | fibers | CNF | CNF | roughness |
| | (μmol/g) | (μmol/g) | (μmol/g) | (μm) |
| CNF_2.5 | 2500 | 1036 ± 41 | 351 ± 11.7 | 0.44 ± 0.02 |
| CNF_3.8 | 3800 | 1364 ± 35 | 326 ± 3.1 | 0.31 ± 0.06 |
| CNF_6.0 | 6000 | 1593 ± 10 | 223 ± 10 | 0.16 ± 0.01 |

The increase in the amount of NaClO led to an increase in the amount of carboxyl groups. Increasing the amount of carboxyl groups increases the repulsion forces between nanofibrils and this facilitates the production of individualized nanofibrils. This was confirmed by the laser profilometry data. The more oxidized the fibers, the higher the nanofibril yield and the smoother the surface of the CNF films. The relatively high roughness profile of CNF_2.5 is due to a major occurrence of residual micrometer-sized fibers. As the oxidation increases, the roughness decreases (see FIG. 1). This is due to the major fraction of individualized nanofibrils that are obtained (see FIG. 2).

AFM analysis revealed that the three samples contain nanofibrils (diameters less than 20 nm) (FIG. 2). The AFM analysis is valuable for providing a comparison between the 3 samples and suggests that sample CNF_2.5 contains relatively thicker nanofibrils (FIG. 2, arrows). This observation is also an indication of a structurally inhomogeneous sample, which confirms the roughness analysis (see Table 1).

The large fraction of individualized nanofibrils of sample CNF_6.0 causes an increase in viscosity of the corresponding gel (see FIG. 3). The three samples show a reduction of viscosity as the speed increases which can be explained from the shear thinning effect. Additionally, the viscosity data indicates that the sample CNF_6.0 has higher viscosity at a given speed, compared to the samples CNF_2.5 and CNF_3.8.

### Example 2—Cytotoxicity and Skin Irritation Potential of CNFs

The cell viability and skin irritation potential of the three CNF samples produced in Example 1 was tested following standardized protocols for assessing medical devices. Six aerogels (20 g/m²) were prepared from each series. The gels were frozen at −20° C. and lyophilized during 24 h, using a Telstar LyoQuest −83 apparatus.

In Vitro EpiDerm Skin Irritation Test:

The skin irritating potential of the samples CNF_2.5, CNF_3.8 and CNF_6.0 was determined by irritation testing according to in vitro skin irritation for medical devices, using the in Vitro EpiDerm™ Skin Irritation Test kit (EPI-200-SIT; MatTek In Vitro Life Science Laboratories, Bratislava, Slovakia) and protocol “In vitro skin irritation test for medical device extracts” v.9.0 final. The test consists of topical exposure of extracts of the test item to the reconstructed human epidermis (RhE) model, followed by a cell viability assay using yellow water-soluble MTT 3-(4,5-dimethylthiazol-2-yl)-2,5-diphenyltetrazoliumbromide which is metabolically reduced to a blue-violet insoluble formazan in viable cells. The number of viable cells correlates to the colour intensity determined by photometric measurements after dissolving the formazan in alcohol.

The RhE tissues were pre-incubated in 6-well plates in assay medium overnight (37±1° C., 5±1% CO₂), after which 100 μL of test item extracts or control samples were added. The positive control was 1% sodium dodecyl sulfate solution (SDS, MatTek In Vitro Life Science Laboratories, Bratislava) in saline and sesame oil, and the negative control was Dulbecco's PBS without Ca²⁺ and Mg²⁺ (GE Healthcare Lifescience HyClone Laboratories, South Logan, Utah). The test item was extracted at 37±1° C. for 72±2 h. After 18 hours of exposure, the tissues were thoroughly rinsed with Dulbecco's PBS without Ca²⁺ and Mg²⁺ (GE Healthcare Lifescience HyClone Laboratories, South Logan, Utah) and incubated in 24-well plates with 1 mg/mL MTT (MatTek In Vitro Life Science Laboratories), for 3 hours (37±1° C. in 5±1% CO₂), The MTT solution was removed, the tissues were immersed in 2-propanol (2 mL/tissue; MatTek), and the plates were shaken for two hours. The absorbance of the extracted formazan was thereafter measured at 570 nm using a spectrophotometer. Skin irritation potential of the test item is predicted if the remaining relative cell viability is below 50%.

Cytotoxicity:

The cytotoxic potential of the samples CNF_2.5, CNF_3.8 and CNF_6.0 was determined by cytotoxicity testing according to ISO 10993-5:2009 Annex C and RISE standard operating procedure SOP KM 11741. The test consists of exposure of extracts of the test item to a sub-confluent monolayer of L929 mouse, followed by a cell viability assay using yellow water-soluble MTT 3-(4,5-dimethylthiazol-2-yl)-2,5-diphenyltetrazoliumbromid which is metabolically reduced to a blue-violet insoluble formazan in viable cells. The number of viable cells correlates to the colour intensity determined by photometric measurements after dissolving the formazan in alcohol.

The test item was extracted at 37±1° C. for 24±2 h in Eagle's Minimum essential medium 1× with Earls balanced salts solution buffered with NaHCO₃ (Gibco Life Technologies) supplemented with nonessential amino acids (Gibco Life Technologies), sodium pyruvate (GE Healthcare HyClone), 5% (v/v) Fetal Bovine Serum (Gibco Life Technologies), 4 mM Stable glutamine (Gibco Life Technologies), 100 IU/mL penicillin and 100 μg/mL streptomycin (GE Healthcare Hyclone) using a ratio of 0.1 g/mL. L929 mouse fibroblasts (ATCC NCTC clone 929: CCL-1) were seeded in a 96-well plate and cultured for at 37±1° C. and 5% CO₂ 24±2 h to form a subconfluent monolayer. 100 μL extracts from test item, positive control (Latex rubber, Gammex 91-325, AccuTech Ansell) and negative control (Thermanox Plastic Coverslips, Art no 174934, Thermo Scientific NUNC), as well as blanks (extraction vehicle to serve as a 100% measure of cell viability) were added to 6 replicate wells. The plate was incubated for 24 hours at 37° C. in 5% CO₂. The extracts were removed and 50 μL of MTT solution was added to each well and the cells were incubated for 2 hours at 37° C. in 5% CO₂. The MTT solution was removed and 100 μL of 2-propanol was added to each well. The plate was shaken rapidly until the formazan from the cells was extracted and formed a homogeneous solution. The absorbance was measured at 570 nm (reference wavelength 650 nm) and the viability of cells was calculated. The test item is considered cytotoxic if the cell viability is below 70%.

Results and Discussion:

Results are presented in Table 2:

TABLE 2

| | Cell viability of | Viability of reconstructed human | |
|---|---|---|---|
| | L929 mouse | epidermis (RhE) (%) | |
| | fibroblasts (%) | Saline | Sesame oil |
| CNF_2.5 | 103.1 ± 3.6 | 92.2 ± 4.3 | 94.4 ± 13.1 |
| CNF_3.8 | 110 ± 3.2 | 93.5 ± 0.7 | 103.9 ± 4.7 |
| CNF_6.0 | 89 ± 1.7 | 98 ± 2.2 | 101.4 ± 9.2 |
| Positive control | 1.3 ± 0.2 | 2.6 ± 0 | 3.1 ± 0 |
| Negative control | 103 ± 2.6 | 100 ± 1.3 | 100 ± 1.3 |

The results confirm that the CNF samples do not have a cytotoxic potential (fibroblast cell viability was greater than 70%, see Table 2). According to criteria given in “In Vitro EpiDerm™ Skin Irritation Test (EPI-200-SIT)” and protocol “In vitro skin irritation test for medical devices” the materials are classified as non-irritant, i.e. the viability of RhE is above the limit to be considered with potential for skin irritation (De Jong et al., Toxicology in Vitro 50, 439-449, 2018). These findings confirm the development of a safe and biocompatible wound dressing material.

### Example 3—Antimicrobial Properties of CNFs

The antimicrobial effect of the CNF gels produced in Example 1 (CNF_2.5, CNF_3.8 and CNF_6.0) on P. aeruginosa was assessed in vitro.

Method:

Overnight culture of P. aeruginosa (ATCC 15692) was set to the final bacterial concentration of 1×10⁷ colony forming units (CFU)/mL using optical density (OD) at 600 nm. 10 μL of the prepared bacterial suspension (1×10⁷ CFU/mL) were mixed with 500 μl CNF gel and incubated at 37° C. for 24 h. 230 μL of the mixture was suspended in 2 mL phosphate buffer (0.05% Triton X-100 in 0.0375 M phosphate) and diluted five times in ten-fold steps. 50 μL from each dilution was spread on horse blood agar plates and incubated overnight at 37° C. The number of CFUs on the blood agar plates was counted and the number of CFUs in the original tube with gel and bacteria mix was calculated. This was defined as bacterial survival after 24 hours treatment. For each gel, 5 replicates were performed, and as negative control 500 μL brain heard infusion medium diluted 100 times in H₂O (BHI100) was used instead of CNF gel.

Results and Discussion:

The results in FIG. 4 confirm a dose-dependent antibacterial effect, i.e. increasing the concentration of CNF from 0.2 to 0.6 wt. % reduced the survival of P. aeruginosa. Additionally, it was found that the antimicrobial properties also depend on the surface charge of the CNF. The results show that increasing the surface charge from 1036 to 1593 μmol/g, reduced the bacterial survival. The reduction of bacterial survival may be attributed to the surface chemistry of the CNFs. Increasing the content of carboxyl groups leads to an increase in the nanofibrillation, i.e. a larger CNF yield is obtained during homogenization. The carboxyl content is expected to increase the repulsion forces between individual nanofibrils in the gel dispersion, thus potentially leading to a charge-dependent distribution of nanofibrils in the liquid medium. The higher the charge density, the more homogenously distributed the nanofibrils and the higher the viscosity (see Table 1 and FIG. 3), and potentially the larger the area the nanofibrils cover on the surface of the bacteria. The aldehyde content may also contribute to cross-link the proteins in the cell wall of the gram-negative bacteria, thus being unable to undertake essential functions. Although not wishing to be bound by theory, we postulate that these characteristics may contribute to limit the bacterial survival and growth.

### Example 4—3D Printing of CNFs

The three CNF grades produced in Example 1 (concentration 0.6 wt. %) were tested for 3D printing.

Method:

3D printing was performed with a Regemat3D printing unit. For each series (CNF_2.5, CNF_3.8 and CNF_6.0), four constructs (dimensions 20 mm×40 mm×2 mm) were printed using a 0.58 printing nozzle. The spaces between the printed tracks were 2 mm×2 mm. The height (2 mm) was composed of 4 printed layers. The flow speed during printing was 3 mm/s. As an additional test of print fidelity, the printing performance of the three CNF grades was assessed. Three replicates (20×40 mm) were printed. The structures were composed of only 1 layer for better assessment of printing performance. The distance between printed tracks was 2 mm. The flow speed was 3 mm/s. Images of the 3D printed structures were acquired immediately after printing with an Epson Perfection V750 PRO scanner, in transmission mode. The applied resolution was 2400 dots per inch. The transmission of light through the optical images was quantified with the ImageJ program (version 1.52h) and is reported as the fraction of light transmitted through the construct, relative to the background.

The 3D printed structures were frozen at −20° C. and lyophilized over 24 hours using a Telstar LyoQuest-83 apparatus. Scanning electron microscopy (SEM) assessment of the freeze-dried samples was performed with a Hitachi SU3500 Scanning Electron Microscope. Gold coating was performed with an Agar Auto Sputter Coater (Agar Scientific, Essex CM24 8GF United Kingdom). Images were acquired in secondary electron imaging (SEI) mode, using 5 kV and 6 mm acceleration voltage and working distance, respectively.

Grids were printed with diameter=20 mm, height=1 mm and composed of two layers. The printing nozzle was 0.58 mm. The flow speed during printing was 3 mm/s. The grids were immersed in CaCl₂ (100 mmol) for at least 24 hours before mechanical assessment with a Tl950 Triboindenter from Bruker (former Hysitron). The nano-indentation parameters were: Conical tip; displacement controlled at peak indentation depth of 2000 nm; 0.125 s loading, 0.4 s holding, 0.125 s unloading (total testing time 0.65 s for one indent). At least 20 reproducible indents on random areas were undertaken, for each sample.

Results and Discussion:

An adequate 3D printing process for the CNFs having a concentration of 0.6 wt. % was achieved, i.e. the deposited tracks did not collapse and 3D constructs could be printed.

Optical images of the 3D constructs (target dimensions 40 mm×20 mm×2 mm) were acquired and the light transmittance was quantified. The printed tracks of the samples CNF_2.5 and CNF_3.8 showed weaker definition compared to CNF_6.0. The CNF_6.0 sample demonstrated a 3D construct with well-defined tracks which is an indication of good print fidelity. Light transmittance through the constructs is shown in FIG. 5. When used as a wound dressing, the transparency facilitates the supervision of wound development.

For the 1 layer structures, the CNF_6.0 sample (having a relatively high viscosity and thus larger fraction of nanofibrils) was found to have particularly good printability, i.e. no major defects were observed on the printed structures.

The results of SEM analysis are presented in FIG. 6. The results indicate pore sizes in the micrometer scale, ranging from roughly 10 μm to 200 μm. A particular characteristic of CNF is the high aspect ratio of individualized nanofibrils, the length in the micrometer-scale, compared to the nanometric cross-sectional dimensions. Facilitated by these characteristics and the shear forces during extrusion, the nanofibrils align in the printing direction. The alignment of individual nanofibrils seems also to affect the self-assembly of the structure after lyophilization. Using computerized gradient analysis based on Sobel operators (Gadalamaria et al., Polymer Composite 14(2): 126-131, 1993) and Yoshigi et al., Cytom Part A 55a(2): 109-118, 2003), we were able to quantify the orientation of the aerogels texture. This is represented by polar plots of azimuthal facets, which indicate the main direction of orientation (Chinga et al., Journal of Microscopy-Oxford 227(3): 254-265, 2007). The more elongated the polar plot is the more pronounced is the orientation in a given direction. The polar plots of structures printed in a horizontal direction are obviously horizontally oriented, compared to the vertically oriented polar plots of structures printed vertically. Samples CNF_2.5 and CNF_3.8 have clear orientation patterns defined by the micrometer-sized surface pores. However, sample CNF_6.0 exhibits a more isotropic texture. The surface texture of CNF_6.0 is composed of flakes/walls of self-assembled nanofibrils. Controlling the orientation of the printed pattern is particularly interesting for scaffolds and tissue engineering to control the growth and proliferation of cells in a given direction.

Table 3 shows the stiffness and hardness (nano-mechanical properties) of the CNF hydrogels (0.6 wt. % concentration):

TABLE 3

| Sample | Elastic modulus (MPa) | Hardness (MPa) |
|---|---|---|
| CNF_2.5 | 2.10 ± 0.33 | 0.21 ± 0.07 |
| CNF_3.8 | 3.17 ± 0.54 | 0.52 ± 0.16 |
| CNF_6.0 | 2.44 ± 0.18 | 0.48 ± 0.09 |

The results yield the level of elastic modulus, i.e. ˜2-3 MPa and hardness (˜0.2-0.5 MPa) of the three set of gels. CNF_3.8 and CNF_6.0 have higher hardness values than CNF_2.5.

Stiffness, the resistance to deformation (in the elastic region) of a material upon an applied force, is important for the mechanotransduction response of cells. For example, cells respond to stiffness of biomaterials by reorganizing the cytoskeleton, affecting the cell spreading, proliferation and migration. Thus, the stiffness of the biomaterial affects the biological behavior of the cells and tissue, which may be important from a wound healing point of view.

Conclusions:

The CNFs are 3D printable and offer the capability to form wound dressings which may be adapted to specific requirements (shape and composition) in the x, y, and z directions. The CNF gels can be cross-linked with Ca²⁺ and easily managed to be applied in a wound situation. The wound dressing is in addition transparent which is expected to facilitate the wound healing management.

### Example 5—Preparation of Oxygenated CNFs and Characterisation

Preparation of Oxygenated CNFs:

The CNFs produced in Example 1 (concentration 0.6 wt. % in water) with three different oxidation levels were denominated CNF_2.5, CNF_3.8 and CNF_6.0 (Table 1). The CNFs were diluted to 0.2 wt. % with purified water (Milli-Q water purifier, Millipore, Molsheim, France). The three grades of CNFs were sterilized in high-pressure steam for 20 minutes (121° C.) in an autoclave (TOMY, Autoclave SX-700E, Tokyo, Japan). The gels were kept at 4° C.

The three grades of CNFs were oxygenated by the OXY BIO System (Oxy Solutions, Oslo, Norway). A detailed description of the oxygenation device and production process is described in WO 2016/071691 (Oxy Solutions AS, Oslo, Norway). The OXY BIO System contains a piping system with venturi where oxygen gas (98%, Praxair, cat no. 500183, Oslo, Norway) and CNFs were mixed. During the production, the corresponding CNF was circulated through the oxygenation device continuously for a minimum of 10 minutes. To confirm if the desired oxygen concentration (>30 mg/l) was achieved under the production, the dissolved oxygen (DO) concentration was measured with Orion RDO Oxygen meter (Orion A323, Thermo Scientific, Massachusetts, USA). The production settings were 3.45 bar (liquid pressure) and 200 ml/min O₂ (oxygen gas flow). The CNF was held cold during the whole production. After the production, oxygenated CNF was filled in glass vials (VWR, Pennsylvania, USA, cat. no. 216-3006) and sealed with aluminium center tear seals (VWR, Pennsylvania, USA, cat. no. 218-2117) and Bromobutyl stoppers (VWR, Pennsylvania, USA, cat. no. WHEAW224100-405).

Characterisation of Oxygenated CNFs:

Viscosity of the oxygenated CNFs was assessed with a Brookfield viscometer (Brookfield DV2TRV). The running parameters were: assessed volume: 200 mL. Temperature: 23° C.±1° C. Spindles: V-71.

Quantification of residual fibers was performed with a Fiber Tester (L&W Fiber Tester Plus, Code 912). The equipment quantifies the amount of residual fibers and fines that are larger than 7 μm. A volume of 40 ml of each CNF dispersion (0.2 wt. %) was prepared and quantified. The analysis was based on the acquisition of more than 7800 images. Two replicates were undertaken for each series. The

CNF dispersions were diluted to 0.1 wt. % and analyzed with a Particle size analyzer (N5 Submicron Particle Size Analyzer, Beckman Coulter), which can determine particle sizes in the range of 3 nm-3 μm.

Results and Discussion:

Brookfield viscosity values of the oxygenated CNFs are shown in FIG. 7. There are two specific trends revealed by the viscosity data: (i) the viscosity decreases as the oxidation increases; and (ii) the oxygenation process decreases the viscosity of the corresponding samples, The reduction in viscosity with increasing oxidation at 0.2 wt. % concentration may be due to the residual fibres and fine materials. Residual fibres are relatively long objects that may contribute to increase the viscosity at low concentration of the dispersion.

In FIG. 8B, the analysis of the dispersion with a nanoparticle analyser shows that the mean object size decreases as the oxidation increases. Additionally, quantification with laser profilometry revealed that the fraction of residual fibres (micrometer-sized) decreases correspondingly. This is confirmed by quantifying a reduction of residual fibres and fines as a function of oxidation (FIG. 8A). Consequently, a higher fraction of relatively long objects may be the factor affecting the increase in viscosity of sample CNF_2.5, at diluted dispersions (0.2 wt. %). The reduction in viscosity with oxygenation may be attributed to mechanical stress of CNFs due to circulation through the OXY BIO System during the oxygenation process. An increased concentration of dissolved oxygen may also contribute to a reduction in viscosity, i.e. oxygen may act as a spacer between the nanofibrils.

### Example 6—Shelf-Life Testing of Oxygenated CNFs

Oxygenated and non-oxygenated CNFs were stored in sealed glass vials at room temperature (22° C.) for 5 weeks. Dissolved oxygen (DO) concentrations were measured at production date (week 0) and 5 weeks later by Winkler titration as previously described (Moen et al., Health Sci. Rep. e57, 2018).

As shown in FIG. 9, no significant differences in DO levels were observed between the three CNF grades (31.2 mg/l for CNF_2.5, 29.6 mg/l for CNF_3.8 and 31.6 mg/l for CNF_6.0). This result demonstrates that CNFs with different surface chemistry and morphology can be oxygenated to approximately the same high levels of DO by the OXY BIO System. After 5 weeks storage, the levels of DO in 0.2 wt % CNF were reduced to 26.9%, 31.1% and 38.0%, respectively. Nevertheless, the DO levels were twice as high as control levels.

### Example 7—Antimicrobial Testing of Oxygenated CNFs

Blinded samples of oxygenated CNF, non-oxygenated CNF and Prontosan wound gel as positive control (Braun Medical AG, Sempach, Switzerland, cat. no. 400515), were evaluated for their antimicrobial effect.

Method:

Overnight culture of P. aeruginosa (ATCC 15692) or S. aureus (ATCC 29213) was set to the final bacterial concentration of 1×10⁷ colony forming units (CFU)/ml using optical density (OD) at 600 nm. 10 μl of the prepared bacterial suspension (1×10⁷ CFU/ml) were mixed with 500 μl gel and incubated at 37° C. for 4/24 h. 230 μl was suspended in 2 ml phosphate buffer (0.05% Triton X-100 in 0.0375 M phosphate) and diluted five times in ten-fold steps. 50 μl from each dilution was spread on horse blood agar plates and incubated over night at 37° C. The number of CFUs on the blood agar plates was counted and the number of CFUs in the original tube with gel and bacteria mix was calculated. This was defined as bacterial survival after 4 and 24 hours treatment. For each gel, 5 replicates were performed, and as negative control 500 μl brain heard infusion medium diluted 100 times in H₂O (BHI100) was used instead of gel.

In a first trial, the bacterial survival of the aerobic bacteria Pseudomonas aeruginosa (P. aeruginosa) was assessed. The quantification of bacterial survival was performed after 4 and 24 hours in order to verify a potential rapid antimicrobial effect after 4 hours. This rapid effect was shown after 4 hours (FIG. 10). Oxygenated CNF_2.5, CNF_3.8 and CNF_6.0 after 4 hours had significantly lower survival of P. aeruginosa (P<0.05, Independent 2-tailed t-test) compared to non-oxygenated CNF_2.5, CNF_3.8 and CNF_6.0, respectively. The results were confirmed after 24 hours. Increasing the charge of the CNFs caused a larger antimicrobial effect and this effect was potentiated by oxygenation.

In a second trial, the bacterial survival of the aerobic bacteria Pseudomonas aeruginosa (P. aeruginosa) and Staphylococcus aureus (S. aureus) after 24 hours were investigated (FIG. 9A-B). The trials started 1-3 weeks after the production of oxygenated CNFs. However, FIG. 9 confirms that the potential reduction of dissolved oxygen in the CNF gels is expected to be minor at the time of assessment. CNF (0.2 wt. %) with increasing oxidation levels (CNF_2.5, CNF_3.8 and CNF_6.0, Table 1) had a significant antimicrobial effect (P<0.05, Independent 2-tailed t-test) compared to BHI100 (negative control) in both trials (FIG. 11A-B).

These results confirm that carboxylated CNF gels have an antimicrobial effect. Oxygenated CNF_2.5 and CNF_6.0 had significantly lower survival of P. aeruginosa (P<0.05, Independent 2-tailed t-test) compared to non-oxygenated CNF_2.5 and CNF_6.0, respectively (FIG. 7A). The difference between CNF_3.8 and oxygenated CNF_3.8 was not significant. Lowest bacterial survival of P. aeruginosa was measured for oxygenated CNF_6.0 (FIG. 11A). These results indicate that the higher oxidation level (ONE_6.0), the better the antimicrobial effect. The effect of CNFs is further potentiated in the presence of high levels of dissolved oxygen. Similar results were observed with the bacteria strain S. aureus (FIG. 11B). The gels CNF_6.0 and CNF_6.0 oxygenated perform similar to the Prontosan gel which is a potent antimicrobial, used as control in this study. It is noted that the gels were diluted to 0.2 wt. % concentration for oxygenation by the OXY BIO system. Previously it has been demonstrated that increasing the concentration of carboxylated ONE increases the antimicrobial effect (Jack et al., Carbohydrate Polymers 157, 1955-1962, 2017). It can be expected that a highly oxygenated gel with a higher concentration of nanofibrils will be a potent antimicrobial agent.

### Example 8—SEM Characterization of Biofilms

In order to shed more light on the mechanism of action of the ONE and oxygenated CNFs, biofilms of S. aureus and P. aeruginosa were grown on pig skin and agar and treated with the ONE gels. The samples were fixed, freeze-dried and assessed with SEM.

Method:

Biofilms of P. aeruginosa (ATCC 15692) or S. aureus (ATCC 29213) were grown on pig skin and agar. After the incubation samples were fixed by 2.5% glutaraldehyde overnight, washed by buffer under agitation twice for 30 min, then fixed in 1% osmium tetroxide overnight, washed by ultrapure water under agitation twice for 30 min, plunge-frozen in liquid propane, and freeze-dried overnight. After, the samples were mounted on microscopy pins and coated by 15 nm of Au/Pt. The imaging was done by Zeiss Supra 40VP SEM, in secondary electrode image mode. The acceleration voltage and working distance were 3 kV and 12 mm, respectively.

Results and Discussion:

The results are presented in FIG. 12 and evidence the mode of action of the CNF. FIG. 12A-B are images of CNF entrapping P. aeruginosa. FIG. 120-D are images of CNF entrapping S. aureus. The nanofibrils appear to form a network which surround and entrap the bacteria. The spatial distribution of carboxylated nanofibrils seems to depend on the oxidation degree (carboxylic groups) and this may facilitate the interaction of the CNF with the bacteria. Additionally, the aldehyde groups encountered on the CNF surface (Table 1) may contribute to anchoring the individual nanofibrils to the proteins in the bacteria cell wall, thus entrapping the microorganisms and limiting their mobility and growth. Individual nanofibrils are playing a specific role on entrapping bacteria and potentially limiting their further mobility and growth (FIG. 12).

### Example 9—Preparation of Oxygenated CNFs Hydrogels

Oxygenated hydrogels containing surface-charged nanofibrils were produced from corresponding oxygenated “CNF liquids” having a low concentration (0.2 wt. % or 0.4 wt. %) of nanofibrils by cross-linking (through the —COO⁻ groups) with Ca²⁺ cations.

Method:

The dissolved oxygen (DO) content in 0.2 wt. % and 0.4 wt. % oxygenated nanocellulose, with or without CaCl₂, was determined to test whether the addition of CaCl₂ changes DO and viscosity. CaCl₂ (50 mM or 100 mM) was added after each nanocellulose was oxygenated. The level of DO was then measured by Winkler titration (triplicate measurements) on production day and 1 month later. Changes in viscosity were visually observed.

Results and Discussion:

Results are presented in FIG. 13. The addition of CaCl₂ to 0.2 wt. % oxygenated nanocellulose resulted in a small reduction in DO−6.9 mg/IlDO and 3.8 mg/l DO for 50 mM CaCl₂ (production day and 1 month later, respectively) and 7.7 mg/l DO and 4.5 mg/l DO for 100 mM CaCl₂ (production day and 1 month later, respectively). The addition of CaCl₂ to 0.4 wt. % oxygenated nanocellulose resulted in a small reduction in DO−2.2 mg/l DO for 50 mM CaCl₂ (1 month later) and 0.7 mg/l DO and 3.6 mg/l DO for 100 mM CaCl₂ (production day and 1 month later, respectively). Cross-linking of the “CNF liquids” with Ca²⁺ was found to increase the viscosity due to cross-linking, but without unduly affecting the oxygenation level.

### Example 10—Preparation of Oxygenated CNFs—Hydrogels

Oxygenated hydrogels containing surface-charged nanofibrils were produced from corresponding oxygenated “CNF liquids” having a low concentration of nanofibrils (0.2 wt. %) by mixing with non-oxygenated CNF gels having a higher CNF content (0.6 wt. %).

Method:

Oxygenated CNF (0.2 wt. %) was mixed with non-oxygenated CNF (0.6 wt. %) to obtain an oxygenated CNF with higher CNF concentration (0.4 wt. %). Details of the materials are set out in Table 4:

TABLE 4

| | Carboxyl | CNF | |
|---|---|---|---|
| Material | content of CNF | concentration | |
| Code | (mmol/g) | (wt. %) | Oxygenation |
| 22_01 | 2.5 | 0.2 | |
| 22_02 | 3.8 | 0.2 | |
| 22_03 | 6.0 | 0.2 | |
| 22_04 | 2.5 | 0.2 | X |
| 22_05 | 3.8 | 0.2 | X |
| 22_06 | 6.0 | 0.2 | X |
| 22_07 | 2.5 | 0.4 | |
| 22_08 | 3.8 | 0.4 | |
| 22_09 | 6.0 | 0.4 | |
| 22_10 | 2.5 | 0.4 | X |
| 22_11 | 3.8 | 0.4 | X |
| 22_12 | 6.0 | 0.4 | X |

Brookfield viscosity of the materials was measured as set out in Example 1.

Results:

The viscosities of the materials are given in FIG. 14. The viscosities of samples 22_01 to 22_06 correspond to the results given in FIG. 5. A significant increase in viscosity of the 0.4 wt. % “gels” was observed.

### Example 11—3D Printing of Ooxygenated CNFs

Oxygenated CNFs (CNF_6.0) having concentrations of 0.2 wt. % and 0.4 wt. % were extruded (i.e. injected) through a 50 ml needle tip with 18G cannula syringe (Braun, Einmal lnjektions-Kanule, 1.20×40 mm BC/SB 18Gx1 ½) to test the potential impact of 3D printing on their oxygen content.

The results in FIG. 15 show that the extrusion process did not lead to a significant loss of oxygen. The reductions in dissolved oxygen (DO) were small and not significant (p value=0.277 for 0.2 wt. % nanocellulose and p value=0.393 for 0.4 wt. % nanocellulose).

Similar experiments were carried out with CNF_2.5 and CNF_3.8 materials having concentrations of 0.2 wt. %. Injection through a 50 ml needle tip with 22G cannula syringe (Braun Sterican®, Einmal lnjektions-Kanule, 0.45×12 mm BULB 26Gx½) resulted in a small reduction in DO which was not significant.

### Example 12—Antibacterial Properties of CNFs

The antibacterial effect of the CNF gels produced in Example 1 (CNF_2.5, CNF_3.8 and CNF_6.0) on P. aeruginosa (ATCC 15692, American Type Culture Collection, Manassas, Va.) and S. aureus (ATCC 29213, American Type Culture Collection, Manassas, Va.) was assessed in vitro.

Method—Determination of Bacterial Survival:

Colonies of P. aeruginosa or S. aureus were cultured on horse blood agar plates (Columbia agar, Oxoid, Basingstoke, UK) supplemented with 5% defibrinated horse blood (Swedish National Veterinary Institute, Uppsala, Sweden), then transferred into 10 ml 3.7% brain heart infusion (BHI) broth (Difco, BD Diagnostics, Franklin Lakes, N.J.) and incubated at +37° C., 250 rpm overnight. The bacterial suspension was centrifuged for 10 minutes at 2000×g. The supernatant was discarded and the pellet was re-suspended in 1 ml 0.037% BHI (BHI medium diluted 100 times in water, BHI100). This suspension was further diluted in BHI100 to reach the final bacterial concentration of 1×10⁸ colony forming units (CFU)/ml, as estimated by measuring optical density at 600 nm. 10 μL of the prepared bacterial suspension (1×10⁸ CFU/ml) were mixed with 500 μl CNF gel and incubated at 37° C. for 24 h. 230 μL of the mixture was suspended in 2 ml phosphate buffer (0.05% Triton X-100 in 0.0375 M phosphate) and diluted five times in ten-fold steps. 50 μL from each dilution was spread on horse blood agar plates and incubated overnight at 37° C. The number of CFUs on the plates were counted and the number of CFUs in the original tube with gel and bacteria mix was calculated and defined as bacterial survival after 24 hours treatment. Each gel was tested three times in three separate blinded trials on P. aeruginosa and in one trial on S. aureus. For each trial 5 replicates were performed, and as a negative control 500 μL BHI100 was used instead of CNF gel.

Method—Swimming Assay:

Luria-Bertani broth supplemented with 0.5% glucose and 0.3% agar was melted in boiling water and then cooled to 45° C. before adding the CNF (6 wt. %) to the melted agar in a 5% v/v mixture as described by Silva et al. (J. Mater. Sci. 54(18), 12159-12170, 2019). The mixture was poured into 55 mm petri dishes (7.5 ml per dish) and was cured for 3 hours with the lid tilted. One sample where the CNF gels were replaced with water was used as control. 5 μL of S. aureus (ATCC 29213) (non-flagellated bacteria) or P. aeruginosa (ATCC 15692) (flagellated bacteria) suspension (1×10⁹ CFU/ ml) was inoculated in the centre of each plate by dipping the pipette tip slightly into the agar. The CNFs and controls were tested in triplicates. The plates were incubated in upright position in aerobic conditions at 37° C. for 9 hours. Digital images were acquired of each agar plate and assessed with the ImageJ program. The images were automatically filtered with a median filter to remove noise and automatically thresholded into binary images to segment the bacteria halo. The Feret's diameter of the bacteria halo was quantified and reported as the degree of swimming of each tested sample.

Results:

The results in FIG. 16 confirm an antibacterial effect of the CNF gels. All samples were significantly different compared to the control. The results in FIG. 17 show the swimming levels of P. aeruginosa in the agar gels containing the CNFs.

### Example 13In Vivo Surgical Site Infection (SSI model)—CNF and Oxygenated CNF

The antimicrobial effect of CNF_3.8 and oxygenated CNF_3.8 as prepared in accordance with Examples 1 and 5, respectively, was determined in vivo and compared to Protonsan® wound gel (obtained from B. Braun, Germany).

Method:

Bacterial preparation: Colonies of S. aureus (ATCC 29213) were cultured on horse blood agar plates (Columbia agar, Oxoid, Basingstoke, UK) supplemented with 5% defibrinated horse blood (Swedish National Veterinary Institute, Uppsala, Sweden), then transferred to 10 ml 3.7% brain heart infusion (BHI) broth (Difco, BD Diagnostics, Franklin Lakes, N.J.) and incubated at +37° C., 250 rpm overnight. The bacterial suspension was centrifuged for 10 minutes at 2000×g. The pellet was re-suspended in 1 ml BHI100 and the suspension was further diluted in BHI100 to reach 2×10⁹ CFU/ml, as estimated by optical density at 600 nm. 8 ml of bacterial suspension were transferred into a 15 ml tube and 3-0 silk sutures (684G, Ethicon, Sollentuna, Sweden) were soaked for 30 minutes in the suspension. The sutures were dried on filter paper at +4° C. and kept at +4° C. until use (a maximum of 4 hours). Approximately 5×10³ cells were adsorbed per cm suture as previously described (Hakansson et al., Antimicrob. Agents Chemother. 58(5), 2982-4, 2014). Animal model: The model used in this study has been published previously (Gisby et al., Antimicrob. Agents Chemother. 44(2), 255-60, 2000; Hakansson et al., Antimicrob. Agents Chemother. 58(5), 2982-4, 2014; McRipley Antimicrob. Agents Chemother. 10, 38-44, 1976; Rittenhouse et al., Antimicrob. Agents Chemother. 50, 3886-3888, 2006) and was modified as described below. All animal experiments were performed after prior approval from the local Ethics Committee for Animal Studies at the Administrative Court of Appeals in Gothenburg, Sweden. The animals were kept in a 12-hours light-dark cycle with free access to water and pellets (Lab For, Lantmännen, Malmö, Sweden), and were cared for in accordance with regulations for the protection of laboratory animals. Female CD1 mice (25-30 g, Charles River, Sulzfeldt, Germany) were anaesthetized with isoflurane (Isobavet, Shering-Plough Animal Health, Farum, Denmark). The back of the mouse was shaved with a clipper, washed with 70% ethanol and a 1 cm full-thickness incision wound was placed centrally on the back of the mouse at the neck region with a scalpel. Approximately 1 cm of the infected suture was placed into the wound and a single nylon suture 5-0 Ethilon*II (EH7800H, Ethicon, Sollentuna, Sweden) was used to close the wound to avoid the mouse from scratching. Buprenorfin (48 μg/kg, Temgesic, Shering-Plough, Brussels, Belgium) was given pre-operatively by intraperitoneal injection for post-surgical pain relief. 24 hours post-infection, 30 μl of placebo or active treatment was applied to the wound with a micropipette. 3 hours later, a second 30-μl treatment was applied to the wound. The placebo and treatment stayed in place in the wound. 2 hours after the last treatment, the mice were euthanized by cervical dislocation and an area of 2×1 cm around the wound (including the whole wound area and surrounding tissue) was excised and homogenized with a rotor stator homogenizer (T10 basic ULTRA-TURRAX, IKA-WerkeGmbH & Co. KG, Staufen, Germany) in 2 ml ice cold BIH100. The homogenate was diluted in six 10-fold steps by transferring 22.2 μl to 200 μl phosphate buffer (0.05% Triton X-100 in 0.0375 M phosphate) in a 96 well plate. 50 μl of each dilution was transferred to horse blood agar plates and incubated at +37° C. overnight. The colonies on the plates containing 30-300 CFU were counted and the number of CFUs/wound was determined.

Results:

Results are shown in FIG. 18.

1. An antimicrobial composition comprising charged cellulose nanofibrils dispersed in an aqueous solution, wherein said solution has a dissolved oxygen content of at least 20 mg/l.

2. A composition as claimed in claim 1, wherein said charged cellulose nanofibrils are present in an amount from 0.1 to 1.0 wt. %, preferably from 0.2 to 0.8 wt. %, e.g. from 0.3 to 0.5 wt. %, based on the total weight of the composition.

3. A composition as claimed in claim 1 or claim 2 which is provided in the form of a liquid or a viscous liquid, preferably having a viscosity in the range from 100 to 9,000 mPa·s, e.g. from 100 to 600 mPa·s when measured using a Brookfield viscometer at 10 rpm, 23° C.

4. A composition as claimed in claim 1 or claim 2 which is provided in the form of a hydrogel, preferably a hydrogel having a viscosity in the range from 10,000 to 20,000 mPa·s when measured using a Brookfield viscometer at 10 rpm, 23° C.

5. A composition as claimed in any one of the preceding claims, wherein said charged cellulose nanofibrils are negatively charged.

6. A composition as claimed in any one of the preceding claims, wherein said charged cellulose nanofibrils are surface-oxidised, preferably TEMPO-oxidised.

7. A composition as claimed in claim 6, wherein the carboxylic acid content of the charged cellulose nanofibrils is in the range from 400 to 1750 μmol/g cellulose, preferably at least about 1000 μmol/g cellulose, e.g. at least about 1400 μmol/g cellulose.

8. A composition as claimed in claim 6 or claim 7, wherein the aldehyde content of the charged cellulose nanofibrils is in the range from 10 to 1700 μmol/g cellulose, preferably from 100 to 400 μmol/g cellulose.

9. A composition as claimed in any one of the preceding claims, wherein the charged cellulose nanofibrils are obtained from wood pulp, preferably softwood pulp, e.g. from Pinus radiata.

10. A composition as claimed in any one of the preceding claims, wherein the average diameter of the cellulose nanofibrils is in the range from 3 to 20 nm and/or wherein the average length of the cellulose nanofibrils is in the range from 5 to 10 μm.

11. A composition as claimed in any one of the preceding claims, wherein said solution has a dissolved oxygen content of from 20 to 100 mg/L oxygen, from 20 to 70 mg/L, from 20 to 60 mg/L, from 25 to 50 mg/L, or from 30 to 40 mg/L.

12. A composition as claimed in any one of the preceding claims which further comprises one or more active substances selected from the group consisting of: antibacterial agents, antifungal agents, antiviral agents, antibiotics, growth factors, cytokines, chemokines, nucleic acids, vitamins, minerals, anaesthetics, anti-inflammatory agents, moisturizers, extracellular matrix proteins, enzymes, stem cells from plants, extracts from eggs and eggshells, botanical extracts, fatty acids, and skin penetration enhancers.

13. A method for the preparation of a composition as claimed in any one of claims 1 to 12, said method comprising the step of combining an aqueous solution having a dissolved oxygen content of at least 20 mg/l with a preparation which contains charged cellulose nanofibrils, preferably wherein said preparation is an aerogel comprising charged cellulose nanofibrils.

14. A method for the preparation of a composition as claimed in any one of claims 1 to 12, said method comprising the following steps:
(i) providing a dispersion of said charged cellulose nanofibrils in an aqueous solution; and
(ii) oxygenating said dispersion.

15. A method as claimed in claim 14, wherein step (ii) comprises the following steps:
introducing a liquid comprising said dispersion into a piping network to form a flow stream;
injecting gaseous oxygen into the flow stream to produce a mixture of said liquid and oxygen bubbles; and
passing the flowing mixture of liquid and gaseous oxygen bubbles through a venturi which is arranged to dissolve the gas into the liquid passing through the venturi.

16. A method as claimed in any one of claims 13 to 15 which further comprises the step of subjecting the resulting composition to cross-linking whereby to increase its viscosity.

17. A composition as claimed in any one of claims 1 to 12 for use as an antimicrobial agent, preferably for use in inhibiting the growth of at least one wound pathogen.

18. A composition as claimed in claim 17 for use in the treatment of a wound, preferably for use in the treatment of a chronic wound, more preferably a wound harbouring one or more bacteria selected from Bacteroides species, Clostridium species, Pseudomonas species, Enterococcus species, Enterobacteriacea species, Bacillus species, Streptococcus species, and Staphylococcus species, e.g. a wound harbouring Pseudomonas aeruginosa and/or Staphylococcus aureus.

19. A composition for use as claimed in any one of claims 1 to 12 in the prevention or treatment of a bacterial biofilm on a body surface, preferably on an external body surface, e.g. on the skin.

20. A wound covering (e.g. a bandage, gauze, patch or absorptive pad) having incorporated therein a composition as claimed in any one of claims 1 to 12.

21. A wound dressing in the form of a hydrogel comprising charged cellulose nanofibrils, wherein said hydrogel has a dissolved oxygen content of at least 20 mg/l.

22. A wound dressing as claimed in claim 21 which is formed by 3D printing.

23. A kit for use in treating a wound, the kit comprising:
(a) a sterilised, sealed container or package containing an antimicrobial composition as claimed in any one of claims 1 to 12;
(b) a wound covering, e.g. a wound dressing, bandage, gauze, patch or absorptive pad; and
optionally (c) printed instructions for use of the components of the kit in the treatment of a wound.

24. A kit for use in treating a wound, the kit comprising:
(a) a sterilised, sealed container or package containing an aerogel comprising charged cellulose nanofibrils;
(b) an oxygenated aqueous liquid (e.g. oxygenated water or oxygenated saline) having a dissolved oxygen content of at least 20 mg/l; and
optionally (c) printed instructions for mixing of the components whereby to form an oxygenated hydrogel and its use in the treatment of a wound.
